(19) United States
(12) Patent Application Publication
(10) Pub. No.: US 2014/0271765 A1
(43) Pub. Date: Sep. 18, 2014

(54) METHODS AND DEVICE FOR TREATING OPIOID ADDICTION

(71) Applicant: Titan Pharmaceuticals, Inc., South San Francisco, CA (US)

(72) Inventor: Rajesh Patel, Redwood City, CA (US)

(73) Assignee: Titan Pharmaceuticals, Inc., South San Francisco, CA (US)

(21) Appl. No.: 14/211,497

(22) Filed: Mar. 14, 2014

Related U.S. Application Data
(60) Provisional application No. 61/799,224, filed on Mar. 15, 2013.

(51) Int. Cl. A61K 9/16; A61K 31/485

(52) U.S. Cl. CPC A61K 9/1635; A61K 31/485
USPC 424/422; 514/279

(57) ABSTRACT

The invention described herein solves the challenges encountered in providing a safe, efficacious, and satisfactory option for the treatment of opioid addiction. Methods and devices of the invention allow a subject to receive an implantable formulation comprising an opioid receptor ligand, buprenorphine, or a metabolite thereof as a treatment for opioid addiction. The invention preempts several difficulties encountered with conventional methods for the treatment of opioid addiction, and by doing so the invention improves treatment adherence, compliance, subject satisfaction, and overall success rate.

### CROSS REFERENCE

This application claims priority to U.S. Provisional Application No. 61/799,224, filed on Mar. 15, 2013, the contents of which is incorporated by reference in its entirety.

### BACKGROUND

Dependence on opioids, in the form of heroin or prescription pain medications, is a significant health concern. Methadone maintenance treatment for opioid dependence reduces morbidity, mortality, and the spread of infectious diseases but is restricted to licensed specialty clinics in the United States, requires frequent clinic visits, and has a high risk of overdose. These issues have led to increased use of buprenorphine as a treatment for opioid addiction, and numerous studies support the efficacy of sublingually-administered buprenorphine. In the United States, buprenorphine can be prescribed in office-based physician practice. However, several concerns exist regarding diversion and nonmedical use of sublingual buprenorphine. Poor treatment adherence, resulting in craving and withdrawal symptoms that increase the likelihood of relapse, is also a concern with sublingual buprenorphine.

### SUMMARY OF THE INVENTION

In some embodiments, the invention provides a method of treating opioid addiction in a subject in need thereof, the method comprising: implanting into the subject a device comprising a particulate of at least one opioid receptor ligand, or a pharmaceutically-acceptable salt thereof, and a pharmaceutically-acceptable carrier, wherein the particulate has a mean particle size of about 5 μM to about 350 μM, and wherein the device releases a therapeutically-effective amount of the opioid receptor ligand or the pharmaceutically-acceptable salt thereof.

In some embodiments, the invention provides a method of treating opioid addiction in a subject in need thereof, the method comprising implanting into the subject a device comprising an opioid receptor ligand or a pharmaceutically-acceptable salt thereof, and a pharmaceutically-acceptable carrier, wherein the device has a tensile strength in a range of about 10,000 g/cm² to about 110,000 g/cm²; and wherein the device releases a therapeutically-effective amount of the opioid receptor ligand, or the pharmaceutically-acceptable salt thereof.

In some embodiments, the invention provides a device comprising: at least one opioid receptor ligand, or a pharmaceutically-acceptable salt thereof, and a pharmaceutically-acceptable carrier, wherein the device has a tensile strength in a range of about 10,000 g/cm² to about 110,000 g/cm²; and wherein, upon implantation of the device in a subject, the device releases a therapeutically-effective amount of the opioid receptor ligand, or the pharmaceutically-acceptable salt thereof to the subject.

In some embodiments, the invention provides a device comprising: a particulate of at least one opioid receptor ligand, or a pharmaceutically-acceptable salt thereof, and a pharmaceutically-acceptable carrier, wherein the particulate has a mean particle size of about 5 μM to about 350 μM, and wherein upon implantation of the device into a subject the device releases a therapeutically-effective amount of the opioid receptor ligand or the pharmaceutically-acceptable salt thereof to the subject.

### BRIEF DESCRIPTION OF THE FIGURES

FIG. 1 illustrates the plasma pharmacokinetics of the mean buprenorphine concentration after device insertion for subjects with 4 or 5 implants in two distinct studies (triangled and circled line markers).

FIG. 2 illustrates the plasma pharmacokinetics of the mean buprenorphine concentration after device insertion for subjects with 4 or 5 implants in an open-label study (triangled line marker), and in a double blind study (diamond line marker). The

FIG. 3 is a schematic of the treatment protocol used in two open label treatment studies.

FIG. 4 illustrates a representative site of implantation of a device of the invention in a human.

FIG. 5 illustrates implantation of a device in the subdermal tissue of a subject.

FIG. 6 illustrates the specifications of a representative applicator used to insert a device of the invention into a subject.

FIG. 7 illustrates the mean and individual plasma buprenorphine concentrations versus time in subjects receiving four Probuphine implants without receiving supplemental (rescue) sublingual buprenorphine (semi-logarithmic scale).

FIG. 8 illustrates the mean and individual plasma norbuprenorphine concentrations versus time in subjects receiving four Probuphine implants without receiving supplemental (rescue) sublingual buprenorphine (semi-logarithmic scale).

FIG. 9 illustrates the chemical structure of: panel a) morphine; panel b) codeine; panel c) oxymorphone; and panel d) oxycodone.

FIG. 10 illustrates the chemical structure of: panel a) hydromorphone; panel b) hydrocodone; panel c) dihydromorphine; and panel d) dihydrocodeine.

FIG. 11 illustrates the chemical structure of: panel a) desomorphine; panel b) ethylmorphine; panel c) heroin; and panel d) metopon.

FIG. 12 illustrates the chemical structure of: panel a) norlevophanol; panel b) levopharnol; panel c) nalbuphine; and panel d) normorphine.

FIG. 13 illustrates the chemical structure of: panel a) myrophine; panel b) benzylmorphine; panel c) phenomorphan; and panel d) phenazocine.

FIG. 14 illustrates the chemical structure of: panel a) nicomorphine; panel b) nalorphine; panel c) buprenorphine; and panel d) butorphanol.

FIG. 15 illustrates the chemical structure of: panel a) eptazocine; panel b) metazocine; panel c) levophenacylmorphan; and panel d) pentzatocine.

FIG. 16 illustrates the chemical structure of: panel a) methadone; panel b) normethadone; panel c) isomethadone; and panel d) dipipanone.

FIG. 17 illustrates the chemical structure of: panel a) dimenoxadol; panel b) dimepheptanol; panel c) propoxyphene; and panel d) dextromoramide.

FIG. 18 illustrates the chemical structure of: panel a) dioxaphetyl butyrate; panel b) norpipanone; panel c) phenadoxone; and panel d) ketobemidone.

FIG. 19 illustrates the chemical structure of: panel a) dimethylthiambutene; panel b) ethylmethylthiambutene; panel c) meptazinol; and panel d) ethoheptazine.

FIG. 20 illustrates the chemical structure of: panel a) meperidine; panel b) hydroxypethidine; panel c) trimeperidine; and panel d) properidine.

FIG. 21 illustrates the chemical structure of: panel a) allylprodine; panel b) alphaprodine; panel c) anileridine; and panel d) clonitazene.

FIG. 22 illustrates the chemical structure of: panel a) proheptazine; panel b) dezocine; panel c) phenoperidine; and panel d) piminodine.

FIG. 23 illustrates the chemical structure of: panel a) narceine; panel b) tramadol; panel c) papaverine; and panel d) tapentadol.

FIG. 24 illustrates the chemical structure of: panel a) diampromide; and panel b) alfentanyl.

FIG. 25 illustrates the chemical structure of: panel a) fentanyl; and panel b) lofentanyl.

FIG. 26 illustrates the chemical structure of: panel a) piritramide; and panel b) bezitramide.

FIG. 27 illustrates the chemical structure of: panel a) sufentanil; and panel b) etonitazene.

### DETAILED DESCRIPTION

Heroin, morphine, and some prescription painkillers, for example, OxyContin™, Vicodin™, and Fentanyl, belong to a class of drugs known as opioids. These drugs act on specific receptors in the brain, which also interact with naturally-produced substances known as endorphins or enkephalins, which are important in regulating pain and emotion. While prescription painkillers are highly beneficial medications when used as prescribed, opiates and opioids as a general class of drugs have noteworthy abuse risks. The treatment of opioid addiction represents a significant clinical and societal challenge, and some of the problematic consequences of opioid addiction are characterized by biological, psychological, and social difficulties.

Several attempts have been made to provide efficacious and safe treatments for opioid addiction, many of which are widely described in the literature. Existing treatments include the prescription of methadone, buprenorphine, naltrexone, diamorphine, and levacetylmethanol. However, strict adherence to pharmacological dosage regiments is a prerequisite to the success of most treatments, and a challenge exists when one prescribes drugs to an individual seeking treatment for substance abuse. Not surprisingly, many existing treatments have only achieved limited success. Compliance is low due to the need for frequent dosing and variable blood levels of the drugs used in the treatment cause withdrawal and cravings, which can lead to a potential relapse.

Furthermore, to prevent abuse from implantable devices, dosages of the active compounds contained within the device can be limited, and the device can be manufactured with an increased breaking strength (resistance to crushing and tearing). A useful implantable device for the treatment of opioid addiction comprises particulates of a compound that is therapeutically-effective and innocuous. The size of the particulates and their plasma rate-of-release are useful properties in formulating a device with a useful drug dissolution profile and minimal potential for misuse.

The device and methods of the invention provide a treatment regimen of opioid dependence that is a significant departure from existing treatments. The invention achieves statistically-significant improvement in adherence to prescribed treatment, non-diversion, and nonmedical uses over the existing treatments designed to target opioid addiction (Example 3). A major advantage of the implantable formulation of the invention is limiting the possibility that devices intended for treatment can be diverted to recreational uses. The present device provides a reduced need for daily supervision and clinical visits, minimizes fluctuations in drug plasma concentrations, improves treatment compliance, and reduces the likelihood of diversion.

In some embodiments, the invention provides a method for treating opioid addiction in a subject in need or want of relief thereof, the method comprising implanting a device comprising a particulate of at least one opioid receptor ligand, and a polymer matrix, to the subject, wherein the device is selected based on the therapeutic effects of the opioid receptor ligand, and wherein the device releases a therapeutically-effective amount of the opioid receptor ligand. The method provides an effective therapeutic regimen for addiction in a subject. Subjects can be of any age, including, for example, elderly adults, adults, adolescents, pre-adolescents, children, toddlers, and infants. Non limiting examples of a subject include humans, dogs, cats, horses, pigs, and mice.

The present invention allows for the selection of the most effective opioid receptor ligand in a specific clinical case. No longer do clinicians and subjects need to be limited by existing treatments, and no longer do clinicians have to remove subjects from a prescribed opioid addiction treatment if the subject has an adverse affect to one particular drug. The invention has been devised to incorporate dosage forms of opioid receptor ligands in a manner that provides a therapeutically-effective dosage of treatment. In some embodiments, the opioid receptor ligand is buprenorphine. In some embodiments, the opioid receptor ligand is norbuprenorphine.

The invention has been devised to provide a therapeutically-effective plasma concentration of buprenorphine, norbuprenorphine, an opioid receptor ligand, or a pharmaceutically-acceptable salt of such compounds. Non-limiting examples of therapeutically-effective plasma concentrations of a device of the invention are illustrated in Examples 2, 3, and 4.

### Device.

An implantable device described herein can be implanted into any mammal, including a human. An implantable device can continuously release a therapeutically-effective dose of an opioid receptor ligand, buprenorphine, a metabolite of buprenorphine, or a pharmaceutically-acceptable salt of the foregoing in vivo over an extended period of time. Implantation of the device can improve compliance with drug dosing regimens and reduce abuse potential. Additionally, a device of the invention can provide a gradual release of the therapeutic agent, providing therapeutically effective plasma levels of an opioid receptor ligand, buprenorphine, a metabolite of burprenorphine or a pharmaceutically-acceptable salt of the foregoing.

Non-limiting examples of device shapes include disk shaped, square or rectangular chip-shaped, cylindrical, and square or rectangular rod-shaped. Shapes can be altered by varying the shape of the extruder (Example 1) used in manufacture, cutting the extruded material, or by injecting extruded or mixed material into a mold. The shape of the device can be modified based on a site in a subject's body in which the device is implanted, the tensile-strength of the device, and other factors. In some embodiments, the device is extruded into 26 mm×2.4 mm implants, weighing 125 mg (approximate dimensions, Example 1).

In some embodiments, the device has a length of about 1 cm to about 10 cm, about 1 cm to about 5 cm, about 1 cm to about 2 cm, about 2 cm to about 3 cm, about 3 cm to about 4 cm, or about 4 cm to about 5 cm. In some embodiments, the device has a mass of about 1 mg to about 10 g, about 10 mg to about 5 g, about 25 mg to about 1000 mg, about 20 mg to about 50 mg, about 50 mg to about 100 mg, about 100 mg to about 150 mg, about 150 mg to about 200 mg, or about 50 mg to about 200 mg. In some embodiments, the device has volume of about 0.01 mL to about 2 mL, about 0.05 mL to about 1 mL, about 0.05 mL to about 0.1 mL, about 0.1 mL to about 0.15 mL, about 0.15 mL to about 0.2 mL, about 0.2 mL to about 0.3 mL, or about 0.05 mL to about 0.3 mL.

Multiple implantable devices can be implanted in a subject. The size of the device and the number of devices implanted can depend upon the rate and duration of the sustained release desired. In some embodiments, the number of devices implanted into a subject can be 1, 2, 3, 4, 5, 6, 7, 8, 9, 10, 11, 12, 13, 14, or 15. In some embodiments, the total mass of all devices implanted is about 80 mg to about 320 g.

In some embodiments, the device comprises a particulate of a compound described herein. In some embodiments, the particulate is in a solid state. In some embodiments the particulate is a non-crystalline solid that lacks the long-range order characteristic of a crystal, and therefore is present in an amorphous state. Amorphous forms of the particulate include, for example, gels, thin films, and nanostructured materials. The average particle size can be measured, for example, by the largest diameter or the smallest diameter of a particle.

In some embodiments the average particle size of the particulate is about 5 μm, about 10 μm, about 15 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 100 μm, about 110 μm, about 120 μm, about 130 μm, about 140 μm, about 150 μm, about 160 μm, about 170 μm, about 180 μm, about 190 μm, about 200 μm, about 210 μm, about 220 μm, about 230 μm, about 240 μm, about 250 μm, about 260 μm, about 270 μm, about 280 μm, about 290 μm, about 300 μm, about 310 μm, about 320 μm, about 330 μm, about 340 μm, about 350 μm, about 360 μm, about 370 μm, about 380 μm, about 390 μm, and about 400 μm.

The device of the invention can have a range of particle sizes. In some embodiments, the average particle size can range from about 5 μm to about 25 μm, from about 25 μm to about 50 μm, from about 50 μm to about 75 μm, from about 75 μm to about 100 μm, from about 100 μm to about 125 μm, from about 125 μm to about 150 μm, from about 150 μm to about 175 μm, from about 175 μm to about 200 μm, from about 200 μm to about 225 μm, from about 225 μm to about 250 μm, from about 250 μm to about 275 μm, from about 275 μm to about 300 μm, from about 300 μm to about 325 μm, from about 325 μm to about 350 μm, from about 350 μm to about 375 μm, from about 375 μm to about 400 μm, from about 400 μm to about 425 μm, from about 425 μm to about 450 μm, from about 450 μm to about 475 μm, or from about 475 μm to about 500 μm.

The device of the invention can have a range of tensile strengths. In some embodiments, the device of the invention can have an average tensile strength of about 10,000 g/cm², about 15,000 g/cm², about 20,000 g/cm², about 25,000 g/cm², about 30,000 g/cm², about 35,000 g/cm², about 40,000 g/cm², about 45,000 g/cm², about 50,000 g/cm², about 55,000 g/cm², about 60,000 g/cm², about 65,000 g/cm², about 70,000 g/cm², about 75,000 g/cm², about 80,000 g/cm², about 85,000 g/cm², about 90,000 g/cm², about 95,000 g/cm², about 100,000 g/cm², about 105,000 g/cm², about 110,000 g/cm², about 115,000 g/cm², about 120,000 g/cm², about 125,000 g/cm², about 130,000 g/cm², about 135,000 g/cm², about 140,000 g/cm², about 145,000 g/cm², about 150,000 g/cm², about 155,000 g/cm², about 160,000 g/cm², about 165,000 g/cm², about 170,000 g/cm², about 175,000 g/cm², about 180,000 g/cm², about 185,000 g/cm², about 190,000 g/cm², about 195,000 g/cm², about 200,000 g/cm², about 205,000 g/cm², about 210,000 g/cm², about 215,000 g/cm², about 220,000 g/cm², about 225,000 g/cm², about 230,000 g/cm², about 235,000 g/cm², about 240,000 g/cm², about 245,000 g/cm², or about 250,000 g/cm².

A device of the invention can have a range of tensile strengths. The tensile strength of a device can range from about 10,000 g/cm² to about 50,000 g/cm², from about 50,000 g/cm² to about 100,000 g/cm², from about 75,000 g/cm² to about 125,000 g/cm², from about 100,000 g/cm² to about 150,000 g/cm², from about 125,000 g/cm² to about 150,000 g/cm², from about 150,000 g/cm² to about 200,000 g/cm², or from about 200,000 g/cm² to about 250,000 g/cm².

An implantable device can be administered by implantation in an individual, and an implantable device can be administered by a physician, a nurse, a nurse practitioner, and any suitable health care provider. The device can be implanted subcutaneously in any of a variety of sites of the body, such as the upper arm, the back, or the abdomen. Multiple implantable devices can be administered, and multiple implantable devices can be administered to different body sites at the same or at different administrations.

A device can have a burst period. A burst period can be a time period of substantially-constant release. A burst period can occur during a period of time following the implantation of the device on a subject. A burst period can occur about 1 hour, about 2 hours, about 6 hours, about 12 hours, about 24 hours, about 36 hours, about 48 hours, about 60 hours, or about 72 hours after implantation. A burst period can be reduced by washing the device prior to implantation with, for example, an alcohol, such as ethanol or isopropanol.

The release rate of a compound herein can be altered by modifying parameters such as the percent drug loading, porosity of the matrix, structure of the implantable device, the hydrophobicity of the matrix, or the number of devices implanted in a subject. A hydrophobic coating or a biodegradable coating can be placed over at least a portion of the device to regulate the rate of release further.

### Methods of the Invention.

The methods and device of the invention provide effective, safe, sustainable, and reliable methods for the treatment of opioid addiction. In some embodiments, an implantable device of the invention comprises: buprenorphine or a pharmaceutically-acceptable salt thereof and a polymer matrix, wherein the implantable device has a tensile strength in a range of about 10,000 g/cm² to about 110,000 g/cm², wherein upon implantation in a human the implant releases a therapeutically-effective amount of buprenorphine or the pharmaceutically-acceptable salt thereof to the human.

A method of treating opioid addiction in a subject in need or want of relief thereof can comprise implanting a device comprising a compound described herein, and a pharmaceutically-acceptable carrier to the subject. A subject can receive one or more implants throughout a specified period of time. A subject can be treated with a method and a device of the invention for at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 21 weeks, at least 22 weeks, at least 23 weeks, or at least 24 weeks.

A therapeutically-effective plasma concentration of a compound of the invention can be provided by one or more devices comprising a compound described herein. A therapeutically-effective plasma concentration of a compound described herein can be provided by implanting no more than one device, no more two devices, no more than three devices, no more than four devices, no more than five devices, no more than six devices, no more than seven devices, no more than eight devices, no more than nine devices, or no more than ten devices on a subject. A therapeutically-effective plasma concentration of a compound of the invention can be provided by implanting at least one device, at least two devices, at least three devices, at least four devices, at least five devices, at least six devices, at least seven devices, at least eight devices, at least nine devices, or at least ten devices on a subject. In some embodiments, four devices of the invention are implanted on a subject. In some embodiments, five devices of the invention are implanted on a subject.

A therapeutically-effective plasma level can be from about 0.1 ng/mL to about 0.5 ng/mL, from about 0.1 ng/mL to about 1 ng/mL, from about 0.1 ng/mL to about 1.5 ng/mL, from 0.1 ng/mL to about 2 ng/mL, from 0.1 ng/mL to about 2.5 ng/mL, from 0.1 ng/mL to about 3 ng/mL, from 0.1 ng/mL to about 3.5 ng/mL, from 0.1 ng/mL to about 4 ng/mL, from 0.1 ng/mL to about 4.5 ng/mL, from 0.1 ng/mL to about 5 ng/mL, from 0.1 ng/mL to about 5.5 ng/mL, from 0.1 ng/mL to about 6 ng/mL, from 0.1 ng/mL to about 6.5 ng/mL, from 0.1 ng/mL to about 7 ng/mL, from 0.1 ng/mL to about 7.5 ng/mL, from 0.1 ng/mL to about 8 ng/mL, from about 0.5 ng/mL to about 0.5 ng/mL, from about 0.5 ng/mL to about 1 ng/mL, from about 0.5 ng/mL to about 1.5 ng/mL, from 0.5 ng/mL to about 2 ng/mL, from 0.5 ng/mL to about 2.5 ng/mL, from 0.5 ng/mL to about 3 ng/mL, from 0.5 ng/mL to about 3.5 ng/mL, from 0.5 ng/mL to about 4 ng/mL, from 0.5 ng/mL to about 4.5 ng/mL, from 0.5 ng/mL to about 5 ng/mL, from 0.5 ng/mL to about 5.5 ng/mL, from 0.5 ng/mL to about 6 ng/mL, from 0.5 ng/mL to about 6.5 ng/mL, from 0.5 ng/mL to about 7 ng/mL, from 0.5 ng/mL to about 7.5 ng/mL, or from 0.5 ng/mL to about 8 ng/mL.

A device of the invention can have a burst period. A burst period can correspond to a blood plasma level of a compound described herein that is provided by a device of the invention after implantation. A burst period can have a duration of from about 1 hour to about 6 hours, from about 1 hour to about 12 hours, from about 1 hour to about 18 hours, from about 1 hour to about 24 hours, from about 1 hour to about 30 hours, from about 1 hour to about 36 hours, from about 1 hour to about 42 hours, from about 1 hour to about 48 hours, from about 1 hour to about 54 hours, from about 1 hour to about 60 hours, from about 1 hour to about 66 hours, from about 1 hour to about 72 hours, from about 1 hour to about 78 hours, from about 1 hour to about 84 hours, from about 1 hour to about 90 hours, or from about 1 hour to about 96 hours.

A burst period can release a plasma level of a compound described herein. A plasma level released during a burst period can be therapeutically-effective or not. A plasma level released during a burst period can range from about 1.0 ng/mL to about 1.5 ng/mL, from about 1.0 ng/mL to about 2.0 ng/mL, from about 1.0 ng/mL to about 2.5 ng/mL, from about 1.0 ng/mL to about 3.0 ng/mL, from about 1.0 ng/mL to about 3.5 ng/mL, or from about 1.0 ng/mL to about 4.0 ng/mL. A plasma level released during a burst period can be about 1.0 ng/mL, 1.5 ng/mL, 2.0 ng/mL, 2.5 ng/mL, 3.0 ng/mL, 3.5 ng/mL, or 4.0 ng/mL.

A device of the invention can be subcutaneously implanted on a subject. In some embodiments, no sutures are required for implantation of the device on a subject. In some embodiments, no sutures are required for removal of the device from a subject. A device can be implanted at a depth of about 2 mm to about 3 mm, about 2 mm to about 4 mm, about 2 mm to about 5 mm, about 2 mm to about 6 mm, about 2 mm to about 7 mm, about 2 mm to about 8 mm, about 2 mm to about 9 mm, about 2 mm to about 10 mm, about 3 mm to about 4 mm, about 3 mm to about 5 mm, about 3 mm to about 6 mm, about 3 mm to about 7 mm, about 3 mm to about 8 mm, about 3 mm to about 9 mm, about 3 mm to about 10 mm, about 4 mm to about 5 mm, about 4 mm to about 6 mm, about 4 mm to about 7 mm, about 4 mm to about 8 mm, about 4 mm to about 9 mm, about 4 mm to about 10 mm, about 5 mm to about 6 mm, about 5 mm to about 7 mm, about 5 mm to about 8 mm, about 5 mm to about 9 mm, about 5 mm to about 10 mm, about 6 mm to about 7 mm, about 6 mm to about 8 mm, about 6 mm to about 9 mm, about 6 mm to about 10 mm, about 7 mm to about 8 mm, about 7 mm to about 9 mm, about 7 mm to about 10 mm, about 8 mm to about 9 mm, about 8 mm to about 10 mm. In some embodiments, the device is implanted 2 mm to 3 mm below the skin of a subject.

A device of the invention can vary in mass. A mass of the device can depend on a ratio of the pharmaceutically-acceptable compound described herein and the pharmaceutically-acceptable carrier. In some embodiments, the mass of the device is about 5% pharmaceutically-acceptable compound and about 95% pharmaceutically-acceptable carrier, about 10% pharmaceutically-acceptable compound and about 90% pharmaceutically-acceptable carrier, about 15% pharmaceutically-acceptable compound and about 85% pharmaceutically-acceptable carrier, about 20% pharmaceutically-acceptable compound and about 80% pharmaceutically-acceptable carrier, about 25% pharmaceutically-acceptable compound and about 75% pharmaceutically-acceptable carrier, about 30% pharmaceutically-acceptable compound and about 70% pharmaceutically-acceptable carrier, about 35% pharmaceutically-acceptable compound and about 65% pharmaceutically-acceptable carrier, about 40% pharmaceutically-acceptable compound and about 60% pharmaceutically-acceptable carrier, about 45% pharmaceutically-acceptable compound and about 55% pharmaceutically-acceptable carrier, about 50% pharmaceutically-acceptable compound and about 50% pharmaceutically-acceptable carrier, about 55% pharmaceutically-acceptable compound and about 45% pharmaceutically-acceptable carrier, about 60% pharmaceutically-acceptable compound and about 40% pharmaceutically-acceptable carrier, about 65% pharmaceutically-acceptable compound and about 35% pharmaceutically-acceptable carrier, about 70% pharmaceutically-acceptable compound and about 30% pharmaceutically-acceptable carrier, about 75% pharmaceutically-acceptable compound and about 25% pharmaceutically-acceptable carrier, about 80% pharmaceutically-acceptable compound and about 20% pharmaceutically-acceptable carrier, about 85% pharmaceutically-acceptable compound and about 15% pharmaceutically-acceptable carrier, about 90% pharmaceutically-acceptable compound and about 10% pharmaceutically-acceptable carrier, or about 95% pharmaceutically-acceptable compound and about 5% pharmaceutically-acceptable carrier.

Devices of the invention can be packaged as a kit. In some embodiments, a kit includes written instructions on the use of the device for treatment of a condition, such as opioid addiction. The written material can be, for example, a label. The written material can suggest conditions and methods of administration. The instructions provide the subject and the supervising physician with the best guidance for achieving the optimal clinical outcome from the administration of the therapy.

### Opioids, Metabolites, and Drug Metabolism.

The methods and device described herein are used to treat different types of opioid addiction. In some embodiments, an opioid receptor ligand such as an opiate, a synthetic opioid, a semi-synthetic opioid, a partial opioid agonist, buprenorphine, a metabolite of buprenorphine, or a pharmaceutically-acceptable salt of the above, is selected as a therapeutic compound for the treatment of opioid addiction.

Non-limiting examples of opioid receptor ligands suitable for use with the present invention include, oxycodone, hydromorphone, morphine, hydrocodone, fentanyl, oxymorphone, codeine, alfentanil, allylprodine, alphaprodine, anileridine, benzylmorphine, bezitramide, buprenorphine, butorphanol, clonitazene, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, heroin, hydroxypethidine, isomethadone, ketobemidone, levorphanol, levophenacylmorphan, lofentanil, meperidine, meptazinol, metazocine, methadone, metopon, myrophine, narceine, nicomorphine, norlevorphanol, normethadone, nalorphine, nalbuphene, normorphine, norpipanone, opium, oxymorphone, papvereturn, pentazocine, phenadoxone, phenomorphan, phenazocine, phenoperidine, piminodine, piritramide, propheptazine, promedol, properidine, propoxyphene, sufentanyl, tapentadol, tilidine or tramadol, a plurality of mu, kappa, sigma and delta opioid receptors and receptor sub-types as well as their pharmaceutically-acceptable salts.

Non-limiting examples of an opioid to which a subject can be addicted to include, oxycodone, hydromorphone, morphine, hydrocodone, fentanyl, oxymorphone, codeine, alfentanil, allylprodine, alphaprodine, anileridine, benzylmorphine, bezitramide, buprenorphine, butorphanol, clonitazene, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, heroin, hydroxypethidine, isomethadone, ketobemidone, levorphanol, levophenacylmorphan, lofentanil, meperidine, meptazinol, metazocine, methadone, metopon, myrophine, narceine, nicomorphine, norlevorphanol, normethadone, nalorphine, nalbuphene, normorphine, norpipanone, opium, oxymorphone, papvereturn, pentazocine, phenadoxone, phenomorphan, phenazocine, phenoperidine, piminodine, piritramide, propheptazine, promedol, properidine, propoxyphene, sufentanyl, tapentadol, tilidine or tramadol, a plurality of mu, kappa, sigma and delta opioid receptors and receptor sub-types as well as their pharmaceutically-acceptable salts.

Opioid receptor ligands can be biotransformed and/or metabolized to yield metabolites that are pharmacologically active. A pharmacologically-active metabolite can have different physiological effects than a parent compound. For example, norbuprenorphine can be considered to have an analgesic effect that can be 2% of the analgesic effect achieved by buprenorphine in rats. Norbuprenorphine can also be considered to have a respiratory-depressant activity in the rat that is approximately 10-times more potent than the respiratory-depressant activity of buprenorphine.

A pharmacologically-active metabolite can have a more potent physiological effect than a parent compound. Certain drugs, such as codeine and tramadol, can produce metabolites with pharmacological activity that can be more potent than the parent drugs, respectively, morphine and O-desmethyltramadol. In some embodiments, the metabolite can be responsible for the therapeutic action of the parent drug.

A metabolite can be, for example, a substance that is a physiological by-product of a parent compound. Five metabolites of buprenorphine have been identified in rats: 1) buprenorphine-glucuronide; 2) norbuprenorphine; 3) norbuprenorphine-glucuronide; 4) 6-O-desmethylbuprenorphine; and 5) 6-O-desmethylbuprenorphine-glucuronide. In some embodiments, norbuprenorphine has been identified as a metabolite of buprenorphine that can provide a safe and efficacious treatment of opioid addiction.

The area under the plasma, serum, or blood concentration versus time curve (AUC) can be a useful tool for calculating the relative efficiency of different drug products. The AUC has a number of important uses in toxicology, biopharmaceutics, and pharmacokinetics. The AUC can be used as a measure of drug exposure in toxicology studies. The AUC can be an important parameter in the comparison of drug products in biopharmaceutics. Drug AUC values can be used to determine other pharmacokinetic parameters, such as clearance or bioavailability. Example 4 describes representative AUC's with the device and methods of the invention.

### Pharmaceutically-Acceptable Salts.

The invention provides the use of pharmaceutically-acceptable salts of any therapeutic compound described herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound of the invention. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, a iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the invention. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrrazole, pipyrrazole, imidazole, pyrazine, or pipyrazine.

In some embodiments, an ammonium salt is a triethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrrazole salt, a pipyrrazole salt, an imidazole salt, a pyrazine salt, or a pipyrazine salt.

Acid addition salts can arise from the addition of an acid to a compound of the invention. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a methanesulfonate (mesylate) salt, an ethanesulfonate salt, a benzenesulfonate salt, a p-toluenesulfonate salt, a citrate salt, an oxalate salt, or a maleate salt.

### Pharmaceutically-Acceptable Carrier.

A pharmaceutically-acceptable carrier can be a substance that improves the delivery and the effectiveness of a compound described herein. A pharmaceutically-acceptable carrier can provide a controlled-release of a compound, decrease drug metabolism, and reduce drug toxicity. A pharmaceutically-acceptable carrier can increase the effectiveness of drug delivery to the target sites of pharmacological actions. A pharmaceutically acceptable carrier can be degradable or non-degradable. Non-limiting examples of pharmaceutically-acceptable carrier can include: a) polymers, including synthetic polymers; b) liposomes; c) microspheres; d) albumin microspheres; e) nanofibers; f) protein-DNA complexes; g) protein conjugates; and h) viral particles.

The present invention provides a device comprising a therapeutic agent combined with a polymer matrix. A polymer matrix can be an innocuous holder of the therapeutic agent or a polymer matrix can have an active function in determining the dissolution profile of the therapeutic agent. In some embodiments, the polymer is adhesive.

A polymer agent, such as ethylene vinyl acetate, can be dissolved in an organic solvent and mixed with a therapeutic agent of choice to obtain a homogenous mixture. Such mixture can be used to release a therapeutic compound slowly and steadily in the circulation of a subject. In some embodiments, the polymer matrix of the invention comprises ethylene vinyl acetate. In some embodiments, the therapeutic agent is buprenorphine.

Various polymer matrixes can be used to prepare the disclosed device, including, for example, silicone, hydrogels such as crosslinked poly(vinyl alcohol) and poly(hydroxy ethylmethacrylate), acyl substituted cellulose acetates and alkyl derivatives thereof, partially and completely hydrolyzed alkylene-vinyl acetate copolymers, unplasticized polyvinyl chloride, crosslinked homo- and copolymers of polyvinyl acetate, crosslinked polyesters of acrylic acid and/or methacrylic acid, polyvinyl alkyl ethers, polyvinyl fluoride, polycarbonate, polyurethane, polyamide, polysulphones, styrene acrylonitrile copolymers, crosslinked poly(ethylene oxide), poly(alkylenes), poly(vinyl imidazole), poly(esters), poly(ethylene terephthalate), polyphosphazenes, and chlorosulphonated polyolefines, and combinations thereof. In some embodiments the polymer comprises ethylene vinyl acetate.

Additionally, a biodegradable, or non-erodible, polymer can be used in a device of the invention. Such device provides significant advantages over existing devices by deviating the need for subsequent removal. Examples of biodegradable polymers include polyesters such as 3-hydroxypropionate, 3-hydroxybutyrate, 3-hydroxyvalerate, 3-hydroxycaproate, 3-hydroxyheptanoate, 3-hydroxyoctanoate, 3-hydroxynonanoate, 3-hydroxydecanoate, 3-hydroxyundecanoate, 3-hydroxydodecanoate, 4-hydroxybutyrate, 5-hydroxyvalerate, polylactide or polylactic acid including poly(d-lactic acid), poly(l-lactic acid), poly(d,l-lactic acid), polyglycolic acid and polyglycolide, poly(lactic-co-glycolic acid), poly(lactide-co-glycolide), poly(ε-caprolactone) and polydioxanone. Polysaccharides including starch, glycogen, cellulose and chitin can also be used as biodegradable materials.

Further non-erodible, biodegradable materials suitable for inclusion in a device of the invention can include, for example, proteins such as zein, resilin, collagen, gelatin, casein, silk, wool, polyesters, polyorthoesters, polyphosphoesters, polycarbonates, polyanhydrides, polyphosphazenes, polyoxalates, polyaminoacids, polyhydroxyalkanoates, polyethyleneglycol, polyvinylacetate, polyhydroxyacids, polyanhydrides, hydrogels including poly(hydroxyethyl methylacrylate), polyethylene glycol, poly(N-isopropylacrylamide), poly(N-vinyl-2-pyrrolidone), cellulose polyvinyl alcohol, silicone hydrogels, polyacrylamides, and polyacrylic acid. In some embodiments, a biodegradable polymer is a co-polymer of lactic and glycolic acid.

To obtain sustained-release of buprenorphine, norbuprenorphine, or an opioid receptor ligand of choice, a substrate comprising the therapeutically active agent can be coated with, for example, a hydrophobic material or a hydrophilic material. Examples of combinations of water insoluble and water soluble materials for a coat can include shellac, polyvinylpyrrolidone, and celluloses. Cellulose has three hydroxyl groups (—OH) per unit glucose ring, and these hydroxyl groups form regular inter and intramolecular hydrogen bonds. Since the hydrogen bonds can form a rigid crystalline structure, unsubstituted celluloses can have a stable structure that is not soluble in water or in organic solvents. However, celluloses that have at least one of the hydroxyl groups in a glucose unit substituted, for example, by etherification, can have amorphous structures due to breakage of hydrogen bonds, and can be soluble in water. Examples of water-soluble cellulose ethers include methylcellulose, hydroxypropylmethylcellulose, hydroxyethylcellulose, hydroxyethylmethylcellulose, and hydroxypropylcellulose. A device of the invention can be coated with water-insoluble materials, water-soluble materials, and combinations thereof.

### Dosages.

The device and method of the invention can release a therapeutically-effective amount of an opioid receptor ligand, buprenorphine, or a metabolite thereof, for an extended period of time after a single administration. A single administration of an opioid receptor ligand, buprenorphine, or a metabolite thereof, includes administration of one or more devices or one or more dosage forms at substantially the same time, including, for example, a single visit to a physician.

A compound described herein can be present in a device in a range of from about 1 mg to about 2000 mg; from about 5 mg to about 1000 mg, from about 10 mg to about 500 mg, from about 50 mg to about 250 mg, from about 100 mg to about 200 mg, from about 1 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 550 mg, from about 550 mg to about 600 mg, from about 600 mg to about 650 mg, from about 650 mg to about 700 mg, from about 700 mg to about 750 mg, from about 750 mg to about 800 mg, from about 800 mg to about 850 mg, from about 850 mg to about 900 mg, from about 900 mg to about 950 mg, or from about 950 mg to about 1000 mg.

A compound described herein can be present in a device in an amount of about 1 mg, about 5 mg, about 10 mg, about 20 mg, about 25 mg, about 50 mg, about 80 mg, about 100 mg, about 150 mg, about 200 mg, about 250 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, or about 2000 mg. In some embodiments about 80 mg of the compound is present in a device.

In some embodiments, the device or devices can release a compound herein, in vivo at a rate of about 1 pg/mL per day to about 20 pg/mL per day, about 100 pg/mL per day to about 500 pg/mL per day, about 100 pg/mL per day to about 200 pg/mL per day, about 200 pg/mL per day to about 300 pg/mL per day, about 300 pg/mL per day to about 400 pg/mL per day, or about 400 pg/mL per day to about 500 pg/mL per day. In some embodiments, the ratio of the average to the standard deviation of the amount of a compound, released each day can be less than about 1, about 0.5, about 0.3, about 0.2, or about 0.1 for a time period of at least 1 month, at least about 2 months, at least about 3 months, or about 1 months to about 6 months after the device or devices are implanted.

A dosage of a compound described herein can provide a plasma concentration of a compound described herein. A concentration can be the amount of drug in a given volume of plasma. A device of the invention can provide a peak plasma concentration (Cₘₐₓ) of a compound described herein after administration. A Cₘₐₓ can be provided by one or more devices of the invention, alone or in combination. A mean Cₘₐₓ can be of about 500 pg/mL, about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, about 11 ng/mL, about 12 ng/mL, about 13 ng/mL, about 14 ng/mL, about 15 ng/mL, about 16 ng/mL, about 17 ng/mL, about 18 ng/mL, about 19 ng/mL, about 20 ng/mL, about 21 ng/mL, about 22 ng/mL, about 23 ng/mL, about 24 ng/mL, about 25 ng/mL, about 26 ng/mL, about 27 ng/mL, about 28 ng/mL, about 29 ng/mL, about 30 ng/mL, about 31 ng/mL, about 32 ng/mL, about 33 ng/mL, about 34 ng/mL, about 35 ng/mL, about 36 ng/mL, about 37 ng/mL, about 38 ng/mL, about 39 ng/mL, about 40 ng/mL, about 41 ng/mL, about 42 ng/mL, about 43 ng/mL, about 44 ng/mL, about 45 ng/mL, about 46 ng/mL, about 47 ng/mL, about 48 ng/mL, about 49 ng/mL, about 50 ng/mL, about 51 ng/mL, about 52 ng/mL, about 53 ng/mL, about 54 ng/mL, about 55 ng/mL, about 56 ng/mL, about 57 ng/mL, about 58 ng/mL, about 59 ng/mL, about 60 ng/mL, about 61 ng/mL, about 62 ng/mL, about 63 ng/mL, about 64 ng/mL, about 65 ng/mL, about 66 ng/mL, about 67 ng/mL, about 68 ng/mL, about 69 ng/mL, about 70 ng/mL, about 71 ng/mL, about 72 ng/mL, about 73 ng/mL, about 74 ng/mL, about 75 ng/mL, about 76 ng/mL, about 77 ng/mL, about 78 ng/mL, about 79 ng/mL, or about 80 ng/mL.

A mean Cₘₐₓ can be of about 50 pg/mL to about 250 pg/mL, from about 250 pg/mL to about 500 pg/mL, from about 500 pg/mL to about 750 pg/mL, from about 750 pg/mL to about 1000 pg/mL, from about 1000 pg/mL to about 1250 pg/mL, from about 1250 pg/mL to about 1500 pg/mL, from about 1500 pg/mL to about 1750 pg/mL, from about 1750 pg/mL to about 2000 pg/mL, from about 2000 pg/mL to about 2250 pg/mL, from about 2250 pg/mL to about 2500 pg/mL, from about 2500 pg/mL to about 2750 pg/mL, from about 2750 pg/mL to about 3000 pg/mL, from about 3000 pg/mL to about 3250 pg/mL, from about 3250 pg/mL to about 3500 pg/mL, from about 3500 pg/mL to about 3750 pg/mL, from about 4000 pg/mL to about 4250 pg/mL, from about 4250 pg/mL to about 4500 pg/mL, from about 4500 pg/mL to about 4750 pg/mL, or from about 4750 pg/mL to about 5000 pg/mL.

The time elapsed between implantation and a maximum plasma concentration provided by a device of the invention can be defined as Tₘₐₓ (the time to reach Cₘₐₓ). A Tₘₐₓ can be from about 1 hour to about 6 hours, from about 1 hour to about 12 hours, from about 1 hour to about 18 hours, from about 1 hour to about 24 hours, from about 1 hour to about 30 hours, from about 1 hour to about 36 hours, from about 1 hour to about 42 hours, from about 1 hour to about 48 hours, from about 1 hour to about 54 hours, from about 1 hour to about 60 hours, from about 1 hour to about 66 hours, from about 1 hour to about 72 hours, from about 1 hour to about 78 hours, from about 1 hour to about 84 hours, from about 1 hour to about 90 hours, or from about 1 hour to about 96 hours.

An average plasma concentration, Cₐᵥₑ, can be provided by one or more devices of the invention for a specified period of time. In some embodiments, a Cₐᵥₑ is preceded by a burst period. An average plasma concentration, Cₐᵥₑ, can be of about 100 pg/mL to about 250 pg/mL, about 250 pg/mL to about 500 pg/mL, about 500 pg/mL to about 750 pg/mL, about 750 pg/mL to about 1000 pg/mL, about 1000 pg/mL to about 1250 pg/mL, about 1250 pg/mL to about 1500 pg/mL, about 1500 pg/mL to about 1750 pg/mL, about 1750 pg/mL to about 2000 pg/mL, about 2000 pg/mL to about 2250 pg/mL, about 2250 pg/mL to about 2750 pg/mL, about 2750 pg/mL to about 3000 pg/mL, about 3000 pg/mL to about 3250 pg/mL, about 3250 pg/mL to about 3500 pg/mL, about 3500 pg/mL to about 3750 pg/mL, about 3750 pg/mL to about 4000 pg/mL, about 4000 pg/mL to about 4250 pg/mL, about 4250 pg/mL to about 4500 pg/mL, about 4500 pg/mL to about 4750 pg/mL, or about 4750 pg/mL to about 5000 pg/mL. In some embodiments, the Cₐᵥₑ is from about 50 pg/mL to about 250 pg/mL. In some embodiments, the Cₐᵥₑ is from about 250 pg/mL to about 500 pg/mL. In some embodiments, the Cₐᵥₑ is from about 250 pg/mL to about 750 pg/mL. In some embodiments, the Cₐᵥₑ is from about 500 pg/mL to about 1000 pg/mL. In some embodiments, the Cₐᵥₑ is from about 500 pg/mL to about 1500 pg/mL.

A device and a method of the invention can provide a plasma concentration of a compound described herein that is defined by a plasma Area Under the Curve (AUC). An AUC can provide a plasma compound concentration-time curve, thereby identifying the exposure of a subject to a drug after implantation of one or more devices. The AUC of a compound described herein implanted with the methods of the invention can range from about 10,000 pg/mL*h to about 30,000 pg/mL*h, from about 25,000 pg/mL*h to about 50,000 pg/mL*h, from about 30,000 pg/mL*h to about 60,000 pg/mL*h, from about 50,000 pg/mL*h to about 75,000 pg/mL*h, from about 60,000 pg/mL*h to about 90,000 pg/mL*h, from about 75,000 pg/mL*h to about 100,000 pg/mL*h, from about 90,000 pg/mL*h to about 120,000 pg/mL*h, or from about 100,000 pg/mL*h to about 125,000 pg/mL*h. The AUC of a device of the invention can be about 10,000 pg/mL*h, about 20,000 pg/mL*h, about 30,000 pg/mL*h, about 40,000 pg/mL*h, about 50,000 pg/mL*h, about 60,000 pg/mL*h, about 70,000 pg/mL*h, about 80,000 pg/mL*h, about 90,000 pg/mL*h, or about 100,000 pg/mL*h.

### EXAMPLES

### Example 1

### A Device Providing an Implantable Formulation to Treat Opioid Addiction

The device comprises an implantable polymeric matrix and an active compound for the treatment of opioid addiction. This example describes an embodiment comprising a polymeric matrix of ethylene-vinyl acetate (EVA) copolymer and Buprenorphine Hydrochloride, extruded into 26 mm×2.4 mm implants, massing 125 mg (approximate dimensions).

Materials and Methods. Reagents: a) milled ethylene vinyl acetate copolymer (EVA, 33% VA) (600 μm), supplied by Southwest Research Institute; b) buprenorphine hydrochloride USP<53 μm; c) buprenorphine hydrochloride USP 53-180 μm, supplied by Sigma-Aldrich™ (sieved at SwRI); c) buprenorphine hydrochloride USP 53-180 μm, supplied by Diosynth™ (sieved at SwRI); d) ethylene vinyl acetate copolymer (EVA, 33% VA) Sigma-Aldrich™; e) 95% alcohol, supplied by Equistar; and f) pre-blended EVA/buprenorphine HCl.

Materials and Methods. Equipment: a) Patterson-Kelley™, blend master lab blender, yoke blender, twin shell, 1-quart; b) Thermo-electron twin screw extruder 16 TC 25:1 TC; c) Tapered 2.40 mm Die TI-01-0804-001; d) Die TI-01-0804-005 2.50 mm (horizontal feed); e) Brabender™ volumetric single screw feeder DSR 28; f) Beta laser mike/accuscan 3, model# LD1010XY-S; g) analytical balance, AT261 delta range Mettler Toledo™, capable of 0.0001 g precision; h) Mitutoyo™ digimatic caliper, CD-6″ C; i) model 610 cold air gun, Pelmar Engineering Ltd; j) Waters 2690/95-2996 PDA Detector (HPLC); k) Gas chromatography instrument (6850 Agilent™); 1) Mettler DL 18, Karl Fisher water determination instrument; m) DiSTek™ 2100B and 2100C, dissolution apparatus; n) Globepharma™ unit dose sampling thief (sample die 0.25 CC) model I; o) KVB™ micro sampler SS, 12 with a 0.2CC head; p) Trol-Mation™ conveyor model: BC-0.75x24-US425-401U-4GN180-RAA; q) VWR™ Forced air oven model: 1350 FMS; r) VEW™ vacuum oven model: 1450 MS; and s) 3M™ ionizing air gun model 980.

Implants comprising particulates of buprenorphine and EVA were produced via hot melt extrusion utilizing a twin co-rotating screw extruder. The implants were washed in 95% (v/v) ethanol to remove surface Buprenorphine HCl to control the initial burst release of drug upon implantation. Development of the device focused on four distinct stages: a) the active ingredient and polymeric carrier were uniformly blended; b) the blended mixture was extruded and cut into implants of uniform weight and diameter; c) the implants were washed to remove excess active ingredient from the surface; and d) the washed implants were dried to remove residual ethanol.

TABLE 1 illustrates the uniformity of a particulate of buprenorphine and EVA obtained under three different blending conditions. The EVA copolymer and Buprenorphine HCl were added to the pre-blended material and tumbled at 25 rpm for 30 minutes.

TABLE 1
(% w/w Buprenorphine HCl)

| | 5 minute blend | 30 minute blend | 60 minute blend |
|---|---|---|---|
| Mean | 78.42 | 77.84 | 75.27 |
| STD Dev | 2.5 | 3.6 | 2.5 |
| RSD | 3 | 5 | 3 |
| Min | 75.69 | 72.36 | 71.63 |
| Max | 80.52 | 81.82 | 78.72 |

TABLE 2 illustrates average particle sizes of particulates of EVA and Buprenorphine HCl obtained under three different conditions.

TABLE 2

| | Specifications | Results | |
|---|---|---|---|
| Particle Size | D(0.1) | 1—10.651 | μm |
| | | 2—10.271 | μm |
| | | 3—10.265 | μm |
| | | Avg: 10.396 | μm |
| Particle Size | D(0.5) | 1—94.639 | μm |
| | | 2—91.762 | μm |
| | | 3—90.847 | μm |
| | | Avg: 92.416 | μm |
| Particle Size | D(0.9) | 1—303.249 | μm |
| | | 2—296.419 | μm |
| | | 3—293.166 | μm |
| | | Avg: 297.611 | μm |

Devices were further evaluated based on appearance, rod diameter, rod length, rod weight, tensile strength, and dissolution parameters described in TABLE 3.

TABLE 3

| Test | Specifications | | |
|---|---|---|---|
| Appearance | | | |
| Rod Diameter | 2.4 +/− 0.24 mm | | |
| Rod Length | 26 +/− 2.6 mm | | |
| Rod Weight | 125 +/− 12.5 mg | | |
| Tensile Strength | Record Results ranging from: Ave = 65678 g/cm² to Ave = 102721 g/cm² | | |
| | Record Results ranging from: | | |
| Dissolution | Hours | % Dissolved Sample 1 | % Dissolved Sample 2 |
| | 4 | 1.9 | 3.5 |
| | 24 | 8.4 | 15.8 |
| | 48 | 15.6 | 26.3 |
| | 120 | 29.2 | 42.1 |
| | 168 | 35.7 | 46.5 |

This process provided a mixture of EVA and Buprenorphine HCl, which, when extruded into devices of approximately 26 mm×2.4 mm in size, massing approximately 125 mg, and implanted into subjects, provided the blood plasma profiles described in Example 2.

### Example 2

### Pharmacokinetic Parameters of Implants for the Treatment of Opioid Addiction in Subjects with Opiate Dependence

Buprenorphine implants were administered to subjects for treatment of opioid dependence. The pharmacokinetics and effectiveness of the implants for the treatment of opiate addiction are illustrated in the following example.

The study was designed as an open-label, sequential dose-group study of 12 subjects (6 subjects per dose group) with DSM-IV-defined opioid dependence, who were in a maintenance treatment program with sublingual buprenorphine. Subjects were switched from a sublingual buprenorphine therapy to treatment with devices of Example 1. Subjects maintained on sublingual buprenorphine 8 mg (1 tablet) daily were switched to 2 device implants placed subcutaneously in one arm for 6 months. Rescue therapy with sublingual buprenorphine was provided to subjects who exhibited inadequate therapeutic control as indicated by their clinical condition.

Prior to insertion of implants and while subjects were receiving sublingual maintenance doses, samples for determination of buprenorphine and norbuprenorphine concentrations were obtained at 1 hour post dose representing approximate peak concentrations, and at 24 hours after the previous dose.

Plasma samples for determination of buprenorphine and norbuprenorphine concentration were obtained at 0, 3, 6, 9, 12, 16, 20, 24, 30, 36, and 48 hours; Days 3, 4, 5, 6, 7, 10, 14, and 21; Weeks 4, 6, 8, 10, 12, 14, 16, 18, 20, 22, and 24 as measured by time of insertion of implants.

FIG. 1 illustrates the mean plasma buprenorphine concentration with the device and methods of the invention during twenty four weeks of study. FIG. 2 illustrates the comparison of the mean plasma buprenorphine concentration with the device and methods of the invention on an open labeled study and a double blind study.

During treatment, changes in the observed plasma concentrations of buprenorphine and norbuprenorphine over time curves were determined. The release rate of buprenorphine initially comprised a time period of substantial constant release, followed by a plateau in the release rate of buprenorphine from the implants.

The time course of buprenorphine concentrations was consistent across subjects with an initial increase over the first 24 hours after insertion and a multi-phase decrease thereafter. The plateau phase was reached within 21 days in all subjects and no subject had a reduction in buprenorphine concentrations by more than 50% from Day 17 to removal. Groups with higher doses, or a higher number of implanted devices, exhibited a more rapid achievement of the plateau phase of drug release.

TABLE 4 shows the summary of buprenorphine pharmacokinetic parameters by subject and dosing group (2 or 4 implants) with summary statistics. The following variables for buprenorphine were determined from the observed data over the time period of device insertion: 1) Cₘₐₓ, maximum concentration; 2) tₘₐₓ, time of maximum concentration; 3) Cₘᵢₙ, minimum concentration; 4) Cₘₐₓ/Cₘᵢₙ, ratio maximum/minimum concentration; 5) t_{1/2}, half-life from Day 21 to the last observation before removal; and 6) Cₐᵥₑ, average concentration from Day 21 to the last observation before removal calculated as AUC/time.

TABLE 4

| | | | | | | Cₐᵥₑ | t_{1/2} | |
|---|---|---|---|---|---|---|---|---|
| | | | | | | Day 21 | Day 21 | C_{1/2} |
| | | | | | | to | to | After |
| | | Cₘₐₓ | tₘₐₓ | Cₘᵢₙ | | removal | removal | removal |
| Subject | Implants | (pg/mL) | (H) | (pg/mL) | Cₘₐₓ/Cₘᵢₙ | (pg/mL) | (weeks) | (H) |
| 1 | 2 | 2630 | 9.05 | 282 | 9.33 | 366 | 90.2 | 30.0 |
| 2 | 2 | 1980 | 16.00 | 237 | 8.35 | 344 | 25.1 | 32.0 |
| 3 | 2 | 2040 | 19.08 | 262 | 7.79 | 363 | 50.3 | NE |
| 4 | 2 | 1490 | 23.83 | 305 | 4.89 | 435 | 25.0 | NE |
| 5 | 2 | 1630 | 20.08 | 213 | 7.65 | 253 | NE | 14.4 |
| 6 | 2 | 2200 | 15.97 | 378 | 5.82 | 446 | NE | 18.6 |
| Mean | | 1995 | 17.34 | 279.5 | 7.31 | 367.8 | 47.7 | 23.8 |
| SD | | 409 | 5.00 | 58.1 | 1.65 | 69.9 | 30.8 | 8.6 |
| Median | | 2010 | 17.54 | 272.0 | 7.72 | 364.5 | 37.7 | 24.3 |
| 7 | 4 | | | | | 886 | 47.8 | NE |
| 8 | 4 | 3460 | 12.0 | 517 | 6.69 | 703 | 83.5 | 15.6 |
| 9 | 4 | 3021 | 16.0 | 567 | 5.33 | 690 | NE | 13.1 |
| 10 | 4 | 2740 | 15.75 | 476 | 5.76 | 547 | 95.9 | 15.8 |
| 11 | 4 | 2970 | 12.0 | 593 | 5.01 | 758 | NE | 9.7 |
| 12 | 4 | 3960 | 24.0 | 509 | 7.78 | 710 | 31.6 | 14.3 |
| Mean | | 3230 | 15.95 | 532.4 | 6.11 | 715.7 | 64.7 | 13.7 |
| SD | | 484 | 4.90 | 47.0 | 1.13 | 109.6 | 30.0 | 2.5 |
| Median | | 3020 | 15.75 | 517.0 | 5.76 | 706.5 | 65.7 | 14.3 |

The half-life for buprenorphine concentrations from Day 21 to removal could not be estimated in 4 subjects as their profiles were practically flat over this time period. The shortest half-life during the plateau phase was 25 weeks. Cₘₐₓ was on average 62% higher in the higher dose group as compared with the lower dose group, whereas Cₐᵥₑ from Day 21 to removal was 93% higher, a close to proportional increase with dose. The buprenorphine terminal half-life after device removal was estimated in 9 of the subjects.

TABLE 5 shows the summary of norbuprenorphine pharmacokinetic parameters by subject and dosing group (2 or 4 implants) with summary statistics. The following variables for norbuprenorphine were determined from the observed data over the time period of device insertion: 1) Cₐᵥₑ, average concentration from Day 21 to the last observation before removal calculated as AUC/time; 2) t_{1/2}, half-life from Day 21 to the last observation before removal.

TABLE 5

| | | Cₐᵥₑ | t_{1/2} |
|---|---|---|---|
| | | Day 21 | Day 21 |
| | | to | to |
| | | removal | removal |
| Subject | Implants | (pg/mL) | (weeks) |
| 1 | 2 | 76.8 | 22.4 |
| 2 | 2 | 239.1 | 401.3 |
| 3 | 2 | 109.9 | 202.4 |
| 4 | 2 | 104.1 | 13.3 |
| 5 | 2 | 100.0 | NE |
| 6 | 2 | 103.8 | NE |
| Mean | | 122.3 | 159.9 |
| SD | | 58.4 | 183.0 |
| Median | | 104.0 | 112.4 |
| 7 | 4 | 390.9 | NE |
| 8 | 4 | 344.2 | 145.8 |
| 9 | 4 | 238.5 | NE |
| 10 | 4 | 441.3 | NE |
| 11 | 4 | 303.8 | NE |
| 12 | 4 | 231.2 | NE |
| Mean | | 324.8 | |
| SD | | 83.5 | |
| Median | | 324.0 | |

### Example 3

### Delivery of Buprenorphine Via an Implantable Delivery System In Vivo: Clinical Report of a Randomized, Placebo and Active-Controlled, Multi-Center Study of Subjects with Opioid Dependence Treated with Devices of the Invention

The efficacy and superiority of the device and methods of the invention versus placebo and previously-available therapies in adult subjects with opioid dependence as defined by the Diagnostic and Statistical Manual of Mental Disorders IV text revision (DSM-IV-TR) was studied. Over Weeks 1 through 24 of out subject treatment, the assessment of thrice-weekly urine toxicology results and illicit drug self-reported data were evaluated.

The study was a randomized, placebo- and active-controlled, multicenter study of the device and methods of the invention in adult subjects with opioid dependence. The following groups were evaluated: Group A (4 devices implanted, blinded); Group B (4 placebo implants, blinded); Group C (12 to 16 mg once daily of sub-lingual buprenorphine). FIG. 3 illustrates a schematic of the treatment protocol used in two open label treatment studies. The implant visit occurred within 14 days of the start of induction. FIG. 4 illustrates a representative site of implantation of a device of the invention in a human. FIG. 5 illustrates implantation of a device in the subdermal tissue.

For groups A and B, implants were inserted in the subject's inner upper arm in a brief, in-office procedure, by implant procedure-certified clinicians. FIG. 6 illustrates the specifications of the applicator used to insert a device of the invention in a subject. The same procedure was performed for implant dose increases. At the end of treatment or at early discontinuation, implants were removed in a brief, in-office procedure by the same certified clinicians. All subjects attended twice-weekly manual-guided drug counseling during study Weeks 1 through 12 and weekly drug counseling during Weeks 13 through 24 with the ability to attend additional sessions within protocol-specified limits. All subjects were eligible for receiving limited supplemental sublingual buprenorphine if they met pre-specified criteria for withdrawal symptoms and cravings or at the investigators discretion. Enrolled subjects were male and female, age 18 to 65 years old who met DSM-IV-TR criteria for current opioid dependence.

The primary analysis was a comparison of the cumulative distribution of the percentage of urine samples negative at Week 24 (Weeks 1 through 24) in the 2 treatment groups by using an exact stratified Wilcoxon rank sum (van Elteren) test with (pooled) site and gender as stratification variables. There was a statistically-significant difference (P<0.0001) between the device and methods of the invention and placebo treatments for the probability of urine samples negative for illicit opioids from Weeks 1 through 24. There was also a statistically-significant difference (P<0.0001) between the device and methods of the invention and placebo in favor of the device and methods of the invention for the probability of urine samples negative for illicit opioids from Weeks 1 through 24 with imputation based on illicit drug self-report data. There was a statistically-significant difference (P<0.0001) between the device and methods of the invention and placebo in favor of the device and methods of the invention for the probability of urine samples negative for illicit opioids from Weeks 1 through 24. Weeks 17 through 24 were characterized by a higher probability of negative urine samples in the group comprising the device and methods of the invention. At the end of treatment or at early discontinuation, implants were removed in a brief, in-office procedure.

### Example 4

### Delivery of Buprenorphine Via an Implantable Delivery System In Vivo: Buprenorphine and Norbuprenorphine Blood Plasma Profile in Subjects without Rescue Medication

Buprenorphine implants were administered to subjects for treatment of opioid dependence. The efficacy results and a tabulation of individual subject data and secondary efficacy analysis for the treatment of opiate addiction are illustrated in the following example.

Buprenorphine concentration data for 61 subjects were measured. FIG. 7 and FIG. 8 display the individual (plotted as individual data points) and the weekly mean (trendline between individual data points) concentrations for buprenorphine and norbuprenorphine on a semi-log scale for subjects with 4 implants who did not receive supplemental buprenorphine. Individual concentrations at Week 0 are prior to implantation and at 12 to 24 hours are after the most recent sublingual buprenorphine treatment. Upon implantation of the device on a subject, some subjects can receive a higher plasma concentration of buprenorphine and norbuprenorphine (“burst effect”) (shown in FIG. 7 and FIG. 8 as a higher concentration of the compound received within 2 weeks of implantation). Within about a few days to about two weeks, the plasma concentration of buprenorphine and norbuprenorphine provided by a device of the invention reaches a plateau. FIG. 7 and FIG. 8 illustrate a plateau of a therapeutically-effective plasma level of buprenorphine and norbuprenorphine provided to a subject for at least 24 weeks.

### Example 5

### Half-Life, Renal Excretion, and Area Under the Plasma Concentration Time Curve (AUC) of Buprenorphine in the Blood Plasma of Subjects with Opiate Dependence Treated with Devices and Methods of the Invention

Buprenorphine implants were administered to subjects for treatment of opioid dependence. The pharmacokinetics and effectiveness of the implants for the treatment of opiate addiction are illustrated in the following example.

The study was designed as an open-label, sequential dose-group study of 12 subjects (6 subjects per dose group) with DSM-IV-defined opioid dependence, who were in a maintenance treatment program with sublingual buprenorphine. Subjects were switched from a sublingual buprenorphine therapy to treatment with devices of the invention. For the 2 implant dose group, subjects maintained on sublingual buprenorphine 8 mg (1 tablet) daily were switched to 2 device implants placed subcutaneously in one arm for 6 months. Rescue therapy with sublingual buprenorphine was provided to subjects who exhibited inadequate therapeutic control as indicated by their clinical condition.

Prior to insertion of implants and while subjects were receiving sublingual maintenance doses, samples for determination of buprenorphine and norbuprenorphine concentrations were obtained at 1 hour postdose, representing approximate peak concentrations, and at 24 hours after the previous dose.

Plasma samples for determination of buprenorphine and norbuprenorphine concentration were obtained at 0, 3, 6, 9, 12, 16, 20, 24, 30, 36, and 48 hours; Days 3, 4, 5, 6, 7, 10, 14, and 21; Weeks 4, 6, 8, 10, 12, 14, 16, 18, 20, 22, and 24 as measured by time of insertion of implants. Additional samples were obtained at 10 and 30 minutes; 1, 2, 4, 6, 9, 12, 24, 36, and 48 hours after device removal.

The exponential decay of the plateau pattern of release of buprenorphine from devices of the invention was measured. The biological half-lives, “alpha half-life” (α-Half Life, hours) and “beta half-life” (β-Half Life, hours), of buprenorphine provided patterns of drug distribution and elimination from plasma circulation. The “terminal half-life” (γ-Half life, weeks) of buprenorphine provided patterns of drug elimination from the system and is described in TABLE 6.

TABLE 6

| | | | | | | Time from |
|---|---|---|---|---|---|---|
| | | | | | Model | implant to |
| | | | | | predicted | reaching 2x |
| | | α-Half | β-Half | γ-Half | concentration | last |
| | | Life | Life | life | at removal | concentration |
| Subject | Implants | (hours) | (hours) | (weeks) | (pg/mL) | (h) |
| 1 | 2 | 9.41 | 109 | 120 | 338 | 203 |
| 2 | 2 | 7.55 | 124 | 29.9 | 263 | 396 |
| 3 | 2 | 4.17 | 206 | 457 | 342 | 259 |
| 4 | 2 | 115 | 3578 | 35.7 | 342 | 313 |
| 5 | 2 | 39.7 | 174 | 131,709 | 252 | 180 |
| 6 | 2 | 2.94 | 52.3 | 350,207 | 447 | 101 |
| Mean | | 12.75 | 133.1 | 160.7 | 330.7 | 242.0 |
| SD | | 15.28 | 59.54 | 201.8 | 70.2 | 104.2 |
| Median | | 7.55 | 124.0 | 77.9 | 340.0 | 231.0 |
| 7 | 4 | 3460 | 12.0 | 517 | 6.69 | 703 |
| 8 | 4 | 3021 | 16.0 | 567 | 5.33 | 690 |
| 9 | 4 | 2740 | 15.75 | 476 | 5.76 | 547 |
| 10 | 4 | 2970 | 12.0 | 593 | 5.01 | 758 |
| 11 | 4 | 3960 | 24.0 | 509 | 7.78 | 710 |
| Mean | | 3230 | 15.95 | 532.4 | 6.11 | 715.7 |
| SD | | 484 | 4.90 | 47.0 | 1.13 | 109.6 |
| Median | | 3020 | 15.75 | 517.0 | 5.76 | 706.5 |

The amount of buprenorphine excreted in the urine (ng), the buprenorphine rate of plasma AUC (pg/mL*h), and the rate of renal clearance were measured and are described in TABLE 7.

TABLE 7

| | | | Amount | | |
|---|---|---|---|---|---|
| | | | excreted | Plasma | Renal |
| | | | in urine | AUC | clearance |
| Subject | Implants | Hours | (ng) | (pg/mL*h) | (mL/min) |
| 1 | 2 | 0-24 | 427 | 45,819 | 0.0065 |
| | | 24-48 | NS | | |
| 2 | 2 | 0-24 | 2015 | 36,285 | 0.0386 |
| | | 24-48 | 759 | 32,760 | 0.0161 |
| 3 | 2 | 0-24 | 627 | 35,917 | 0.0121 |
| | | 24-48 | NS | | |
| 4 | 2 | 0-24 | 1528 | 19,591 | 0.0552 |
| | | 24-48 | 1643 | 31,770 | 0.0359 |
| 5 | 2 | 0-24 | 1998 | 21,948 | 0.0632 |
| | | 24-48 | 5031 | 30,840 | 0.1133 |
| 6 | 2 | 0-24 | 1706 | 41,836 | 0.0283 |
| | | 24-48 | 2618 | 36,510 | 0.0498 |
| Mean | | | | | 0.0418 |
| SD | | | | | 0.0313 |
| Median | | | | | 0.0373 |
| 7 | 4 | 0-24 | 583 | NC | NC |
| | | 24-48 | 894 | NC | NC |
| 8 | 4 | 0-24 | 1863 | 58,490 | 0.0221 |
| | | 24-48 | 1266 | 45,240 | 0.0194 |
| 9 | 4 | 0-24 | 1030 | 49,165 | 0.0146 |
| | | 24-48 | 1058 | 35,670 | 0.0206 |
| 10 | 4 | 0-24 | 560 | 44,431 | 0.0088 |
| | | 24-48 | 6304 | 40,410 | 0.1083 |
| 11 | 4 | 0-24 | 1699 | 48,998 | 0.0241 |
| | | 24-48 | 2490 | 49,800 | 0.0347 |
| 12 | 4 | 0-24 | 4310 | 68,715 | 0.0436 |
| | | 24-48 | 3906 | 72,000 | 0.0377 |
| Mean | | | | | 0.0334 |
| SD | | | | | 0.0284 |
| Median | | | | | 0.0231 |

NS: no sample.
NC: Not calculated.

The amount of norbuprenorphine excreted in the urine (ng), the norbuprenorphine rate of plasma AUC (pg/mL*h), and the rate of renal clearance were measured and are described in TABLE 8.

TABLE 8

| | | | Amount | | |
|---|---|---|---|---|---|
| | | | excreted | Plasma | Renal |
| | | | in urine | AUC | clearance |
| Subject | Implants | Hours | (ng) | (pg/mL*h) | (mL/min) |
| 1 | 2 | 0-24 | 37,290 | 24,379 | 1.062 |
| | | 24-48 | NS | | |
| 2 | 2 | 0-24 | 105,400 | 49,780 | 1.470 |
| | | 24-48 | 35,640 | 46,200 | 0.536 |
| 3 | 2 | 0-24 | 59,695 | 24,287 | 1.707 |
| | | 24-48 | NS | | |
| 4 | 2 | 0-24 | 105,000 | 27,032 | 2.697 |
| | | 24-48 | 89,900 | 24,618 | 2.536 |
| 5 | 2 | 0-24 | 135,900 | 24,987 | 3.777 |
| | | 24-48 | 77,415 | 25,638 | 2.097 |
| 6 | 2 | 0-24 | 65,142 | 18,773 | 2.410 |
| | | 24-48 | 63,840 | 12,846 | 3.451 |
| Mean | | | | | 2.174 |
| SD | | | | | 1.017 |
| Median | | | | | 2.254 |
| 7 | 4 | 0-24 | 32,780 | NC | NC |
| | | 24-48 | 35,300 | NC | NC |
| 8 | 4 | 0-24 | 163,750 | 87,440 | 1.300 |
| | | 24-48 | 146,250 | 76,920 | 1.320 |
| 9 | 4 | 0-24 | 59,186 | 85,055 | 0.483 |
| | | 24-48 | 55,440 | 60,930 | 0.632 |
| 10 | 4 | 0-24 | 18,900 | 74,000 | 0.177 |
| | | 24-48 | 235,200 | 70,290 | 2.324 |
| 11 | 4 | 0-24 | 56,640 | 41,040 | 0.958 |
| | | 24-48 | 71,900 | 29,250 | 1.707 |
| 12 | 4 | 0-24 | 128,370 | 23,182 | 3.846 |
| | | 24-48 | 115,520 | 22,989 | 3.490 |
| Mean | | | | | 0.0334 |
| SD | | | | | 0.0284 |
| Median | | | | | 0.0231 |

NS: no sample.
NC: Not calculated.

### Example 6

### Pharmacokinetics of Buprenorphine Release from Subcutaneous Implants in Dogs after Heat Application

Plasma samples were harvested and pharmacokinetic analyses of the obtained plasma buprenorphine concentration-time data were subsequently conducted. No consistent changes in plasma buprenorphine exposure were observed when external heat was applied for 8 hours directly after implantation (Groups 1 and 2) or when reapplied for 8 hours at 5 weeks after implantation (Group 2). The plasma buprenorphine Cₘₐₓ and AUC₀₋₄₈ values ranged from 6.11 to 9.81 ng/mL and 207 to 355 ng·hr/mL, respectively, when heat was applied to the implant site for 8 hours on Day 1 and from 8.64 to 11.3 ng/mL and 286 to 401 ng·hr/mL, respectively, when heat was not applied to the implant site (PK Phase 1). During Week 5 (PK Phase 3) post implantation, the steady-state plasma buprenorphine concentrations ranged from 2.61 to 5.42 ng/mL when heat was applied to the implant site for 8 hours and from 2.73 to 4.84 ng/mL when heat was not applied to the implant site. Norbuprenorphine plasma concentrations were generally below the limit of quantitation and pharmacokinetic analysis was not undertaken.

Experimental design and procedures. Dogs were assigned to two groups for this study. At designated times following implantation, blood and skin surface temperatures were collected. The group designations, number of animals, target dose level, and target dose volume were as follows:

| | | | | | Target Dose |
|---|---|---|---|---|---|
| | PK | Number | Dose | Heat | Level |
| Group | Phase | of Males | Route^{a} | Application | (mg/animal)^{b} |
| 1 | 1 | 6 | SC | No | 400 |
| 2 | 1 | 6 | SC | Yes^{c} | 400 |
| 2 | 2 | 6 | SC | No | NA |
| 2 | 3 | 6 | SC | Yes^{c} | NA |

NA Not applicable
PK Pharmacokinetic
SC Subcutaneous
Note:
There were 4 weeks between PK Phases 1 and 2, and 1 week between PK phases 2 and 3.
^{a}Implants were inserted subcutaneously on Day 1 (PK Phase 1); Group 1 implants were removed following completion of PK Phase 1, and Group 2 implants were removed following completion of PK Phase 3.
^{b}Each animal in Groups 1 and 2 (n = 12) received one dose of 5 implants (each implant containing 80 mg buprenorphine HCl) on Day 1 (PK Phase 1). Group 1 animals were assessed for plasma PK during Phase 1, while Group 2 animals were assessed for plasma PK in Phases 1, 2, and 3.
^{c}A pre-activated heat patch was applied to the dose site for 8 hours; skin-surface temperature beneath the heat patch was measured at specified times.

Test animals and housing. Twelve male purebred Beagle dogs were maintained and monitored for good health. Animals were acclimated to the study room for 12 days prior to dose administration. On the day of dosing, the animals weighed 10.2 to 12.1 kg and were 6 to 7 months of age. All animals were housed in individual, stainless steel cages during acclimation and the test periods. Food was provided ad libitum, unless otherwise specified under dosing procedures. Diets were supplemented with canine treats. Additionally, per veterinary directive, canned dog food was provided to all animals beginning on Day 3 through the duration of the study. Water was provided fresh daily, ad libitum. Environmental controls for the animal room were set to maintain a temperature of 20 to 26° C., a relative humidity of 50±20%, and a 12-hour light/12-hour dark cycle. As necessary, the 12-hour dark cycle was interrupted to accommodate study procedures.

Identification and animal selection. Animals were assigned to study based on body weight, overall health, and other parameters as applicable. Each animal was uniquely identified by an implantable microchip identification device (IMID).

Dose administration. Animals were fasted overnight prior to implantation. Implants were inserted and removed according to a study specific procedure. Five test article implants were inserted subcutaneously using insertion applicators provided by the sponsor in the dorsal-scapular region of each animal on Day 1 of pharmacokinetic (PK) Phase 1. Implants were inserted in a fan-like configuration from a single incision point; the incision was sutured closed following implantation and the location of each implant was recorded. The time of dose administration was recorded as the time the fifth implant was inserted. Following the completion of PK Phase 1 (Group 1) or PK Phase 3 (Group 2), implants were surgically removed and stored at approximately −20° C. prior to disposal.

Heat patch placement. For Group 2 in PK Phases 1 and 3, external heat was applied to the implant area of each animal via a commercially available heat patch immediately after implantation (within 5 minutes for Phase 1 time 0) or immediately following the Phase 3 time 0 blood collection (within 5 minutes). Prior to placement on the animal, the air-activated heat patch was opened and allowed to warm until the temperature reached 40±1° C. A calibrated thermal sensor was adhered to the dose site prior to placement of the heat patch. Prior to use, the sensors were calibrated by the from approximately 30° C. to approximately 45° C. using 3 test points per sensor; calibration results are maintained in the raw data. Following the 8-hour blood collection and temperature measurement for each applicable phase, the heat patch was removed immediately (within 2 minutes). Animals were jacketed when calibrated thermal sensors were in place.

Observation of animals. At least twice daily (a.m. and p.m.), animals were observed for mortality and signs of pain and distress. Cageside observations for general health and appearance were done once daily. Any observations (e.g., lethargy, salivation, emesis, loss of appetite, implant site erythema, redness, or irritation) were noted along with the date and time of the observation. Animals were weighed at arrival, the day prior to implantation, predose on the day of implantation, the day after implantation, and weekly thereafter.

Sample collection, Blood and Plasma. For PK Phase 1, blood (approximately 2 mL) was collected from a jugular vein into tubes containing K₂EDTA anticoagulant from all animals predose and at 2, 4, 6, 8, 10, 12, 16, 20, 24, 30, 36, 42, and 48 hours post-implantation. For PK Phase 2, at four weeks following implant insertion (approximately 672 hours post-implantation), blood (approximately 2 mL) was collected from a jugular vein into tubes containing K₂EDTA anticoagulant from all animals at approximately the same time of day as the PK Phase 1 dose administration (time 0) and at 6, 12, 18, and 24 hours following the time 0 collection. For PK Phase 3, at five weeks following implant insertion (approximately 840 hours post-implantation), blood (approximately 2 mL) was collected from a jugular vein into tubes containing K₂EDTA anticoagulant from all animals at approximately the same time of day as the PK Phase 1 dose administration (time 0) and at 2, 4, 6, 8, 10, 12, 16, 20, 24, 30, 36, 42, and 48 hours following the time 0 collection.

Sample collection, skin surface temperature. For PK Phases 1 and 3 (Group 2), temperature of the skin surface beneath the heat pad applied at the implant site was measured for each animal using a calibrated thermal sensor immediately post-implantation (within 5 minutes) prior to heat pad placement (or at time 0 for PK Phase 3, prior to heat pad placement) and at 2, 4, 6, 8, 10, and 12 hours post-implantation (or post time 0 for PK Phase 3).

Sample identification, handling, storage, and shipment. Samples were uniquely identified with, but not limited to, study number, animal identification number, and sample type to indicate origin and collection time. Blood was maintained on a chilled cryorack prior to centrifugation to obtain plasma. Samples were centrifuged at approximately 3000 rpm for approximately 10 minutes in a centrifuge set to maintain 2 to 8° C. Centrifugation began within 1 hour of collection. Plasma was harvested and maintained on dry ice prior to storage at approximately −70° C.

Pharmacokinetic analysis. The following pharmacokinetic parameters were estimated, whenever possible: determination of maximum concentration (Cₘₐₓ), time to maximum concentration (Tₘₐₓ), area under the curve from the time of implantation (hour 0) to hour 48 after implantation (AUC₀₋₄₈, calculated for Phase 1), area under the concentration-time curve from hour 0 to hour 24 (AUC₀₋₂₄, calculated for Phases 2 and 3), average plasma concentration at 1 month after implantation from hour 0 to hour 24, without heat application (Cₛₛ₁, calculated for Phase 2), average plasma concentration at 1 month after implantation from hour 0 to hour 8, during the 8-hour heat application (Cₛₛ₂, calculated for Phase 3), and average plasma concentration from hour 10 to hour 48, after removal of heat at hour 8(Cₛₛ₃, calculated for Phase 3).

Disposition of test article. Any remaining test article pouches were destroyed following sponsor-provided authorization. Implants recovered from animals were stored at approximately −20° C. Recovered implants were then discarded following written authorization from the sponsor.

Disposition of raw data, records, samples, and the final report. The original signed protocol, the original signed report, the study correspondence, and raw data captured on durable media will be archived in the storage facilities. All other raw data, documentation, and records will be archived in the storage facilities until shipped to another site. Archival of any data generated or samples remaining at the test site is the responsibility of the test site.

### Results and Discussion:

Acclimation. All animals appeared clinically healthy throughout acclimation.

Body weights. Body weight declined in all animals from Day 1 to Day 2. For Group 1, mean body weight declined approximately 3% from Day 1 to Day 2 in Phase 1. For Group 2, mean body weight declined approximately 4% from Day 1 to Day 2. Mean body weights increased or remained steady throughout the remaining duration of the study, as applicable.

Sample collections. In accordance with the protocol and Covance SOPs, all collections were made within the acceptable ranges. A summary of acceptable time ranges follows:

| Scheduled Collection Time | Acceptable Time Range |
|---|---|
| 0-15 minutes | ±1 minute |
| 16-30 minutes | ±2 minutes |
| 31-45 minutes | ±3 minutes |
| 46-60 minutes | ±4 minutes |
| 61 minutes-2 hours | ±5 minutes |
| 2 hours 1 minutes-8 hours | ±10 minutes |
| >8 hours-24 hours | ±20 minutes |
| >24 hours | ±60 minutes |

Skin surface temperature. Overall, skin surface temperatures in Phases 1 and 3 were similar, and individual animal variability was low for each measured time point Skin temperatures prior to heat patch placement ranged from 30.4 to 36.3° C., respectively for Phases 1 and 3 (mean of 33.0° C. and 34.1° C., respectively). Variability of the skin surfaces temperatures collected prior to heat patch placement may be attributed to lack of full equilibration of the thermal chip to the skin surface, as the chips were exposed to the ambient air prior to placement on the skin. For all animals, skin surface temperatures increased following placement of the heat patch in Phases 1 and 3. From 0 to 2 hours post patch placement, mean skin surface temperatures increased from 33.0 to 41.3° C. in Phase 1 and from 34.1 to 40.2° C. in Phase 3. During the time the heat patch was in place, individual and group mean skin surface temperatures were consistent and steady for all animals in Phases 1 and 3 with one exception in which, skin surface temperature gradually declined from 39.5° C. at 2 hours postdose to 37.9° C. at 8 hours postdose (Phase 1 only). Individual skin surface temperatures ranged from 37.9 to 42.2° C.; mean temperatures ranged from 39.9 to 41.3° C. Heat patches were removed immediately following completion of the 8 hour sample collections. Two hours after removal (10-hour postdose) mean skin surface temperatures decreased to 35.6 and 37.3° C. for Phases 1 and 3, respectively. At 12 hours postdose, mean skin surface temperatures were similar to the 10-hour measurements, with mean temperatures of 35.9 and 37.0° C. for Phases 1 and 3, respectively.

Animal observations. All animals appeared healthy prior to dosing and throughout the duration of the study, with the following exceptions. The initial lethargy, hypothermia, and excessive salivation observed in some animals following implantation are consistent with the pharmacokinetic-observed initial peak release of buprenorphine from the implants. In addition, the initial lethargy, hypothermia, and excessive salivation observed in some animals shortly following implantation may be related to the interaction of Midazolam, administered to sedate the animals prior to implantation, with the pharmacokinetic-observed initial peak release of buprenorphine from the implants. The combination of benzodiazepines and buprenorphine have been reported to alter the usual ceiling effect on buprenorphine-induced respiratory depression.

Buprenorphine concentrations and pharmacokinetics. As previously discussed, for Group 2, during PK Phases 1 and 3, a commercially available heat patch was warmed to 40±1° C. prior to placement, and applied to the dose site for 8 hours following implantation (or at Phase 3 time 0, as applicable). Skin surface temperatures increased from a mean of 33.0° C. (Phase 1) and 34.1° C. (Phase 3) prior to heat patch application, to a mean temperature range of 40.5 to 41.3° C. (Phase 1) and 39.9 to 40.2° C. (Phase 3) during the time of heat patch placement (2-8 hours post-heat patch placement).

The mean concentration-time profiles showed that exposure to buprenorphine in the plasma in PK Phase 1 was similar in animals after 8 hours of heat application to the implant site (Group 2; the mean plasma buprenorphine concentration was 3.69 ng/mL and ranged from 1.45 to 5.63 ng/mL) when compared to animals that did not have 8 hours of heat applied to the implant site (Group 1; the mean plasma buprenorphine concentration was 4.18 ng/mL and ranged from 2.41 to 6.4 ng/mL). The plasma buprenorphine Cₘₐₓ and AUC₀₋₄₈ values were 7.96 ng/mL (ranging from 6.11 to 9.81 ng/mL) and 274 ng·hr/mL (ranging from 207 to 355 ng·hr/mL), respectively, when heat was applied to the implant site for 8 hours on Day 1, and were 9.89 ng/mL (ranging from 8.64 to 11.3 ng/mL) and 343 ng·hr/mL (ranging from 286 to 401 ng·hr/mL), respectively, when heat was not applied to the implant (PK Phase 1).

The results in PK Phases 2 and 3 showed that mean steady-state plasma buprenorphine concentration (Cₛₛ₂) was 4.37 ng/mL (ranging from 2.61 to 5.42 ng/mL) following 8 hours of heat application to the implant site five weeks after implantation (PK Phase 3). These results are generally similar to the mean steady-state concentration (Cₛₛ₃) of 3.86 ng/mL (ranging from 2.73 to 4.84 ng/mL) following the removal of external heat (PK Phase 3) and the mean steady state concentration (Cₛₛ₁) of 3.90 ng/mL (ranging from 3.01 to 4.77 ng/mL) in Week 4 post-implantation when heat was not applied to the implant site (PK Phase 2).

PK analysis was not performed for norbuprenorphine data as these plasma concentrations were generally below the limit of quantitation.

Conclusions. The purpose of this GLP study was to determine the pharmacokinetics of buprenorphine release from subcutaneous Probuphine® implants in dogs following external application of heat. Concentrations of buprenorphine and norbuprenorphine in plasma obtained at specific time points were determined using a validated GLP bioanalytical method and pharmacokinetic parameters were determined. No consistent changes in plasma buprenorphine exposure were observed when external heat was applied for 8 hours directly after implantation or when reapplied at the time of steady state release 5 weeks after implantation. Norbuprenorphine plasma concentrations were generally below the limit of quantitation and pharmacokinetic analysis was not undertaken.

Various opioids with different chemical structures can be used with a device and method of the invention. FIG. 9 illustrates the chemical structure of panel a) morphine, panel b) codeine, panel c) oxymorphone, and panel d) oxycodone. FIG. 10 illustrates the chemical structure of panel a) hydromorphone, panel b) hydrocodone, panel c) dihydromorphine, and panel d) dihydrocodeine. FIG. 11 illustrates the chemical structure of panel a) desomorphine, panel b) ethylmorphine, panel c) heroin, and panel d) metopon. FIG. 12 illustrates the chemical structure of panel a) norlevophanol, panel b) levopharnol, panel c) nalbuphine, and panel d) normorphine. FIG. 13 illustrates the chemical structure of panel a) myrophine, panel b) benzylmorphine, panel c) phenomorphan, and panel d) phenazocine. FIG. 14 illustrates the chemical structure of panel a) nicomorphine, panel b) nalorphine, panel c) buprenorphine, and panel d) butorphanol. FIG. 15 illustrates the chemical structure of panel a) eptazocine, panel b) metazocine, panel c) levophenacylmorphan, and panel d) pentzatocine. FIG. 16 illustrates the chemical structure of panel a) methadone, panel b) normethadone, panel c) isomethadone, and panel d) dipipanone. FIG. 17 illustrates the chemical structure of panel a) dimenoxadol, panel b) dimepheptanol, panel c) propoxyphene, and panel d) dextromoramide. FIG. 18 illustrates the chemical structure of panel a) dioxaphetyl butyrate, panel b) norpipanone, panel c) phenadoxone, and panel d) ketobemidone. FIG. 19 illustrates the chemical structure of panel a) dimethylthiambutene, panel b) ethylmethylthiambutene, panel c) meptazinol, and panel d) ethoheptazine. FIG. 20 illustrates the chemical structure of panel a) meperidine, panel b) hydroxypethidine, panel c) trimeperidine, and panel d) properidine. FIG. 21 illustrates the chemical structure of panel a) allylprodine, panel b) alphaprodine, panel c) anileridine, and panel d) clonitazene. FIG. 22 illustrates the chemical structure of panel a) proheptazine, panel b) dezocine, panel c) phenoperidine, and panel d) piminodine. FIG. 23 illustrates the chemical structure of panel a) narceine, panel b) tramadol, panel c) papaverine, and panel d) tapentadol. FIG. 24 illustrates the chemical structure of panel a) diampromide, and panel b) alfentanyl. FIG. 25 illustrates the chemical structure of panel a) fentanyl, and panel b) lofentanyl. FIG. 26 illustrates the chemical structure of panel a) piritramide, and panel b) bezitramide. FIG. 27 illustrates the chemical structure of panel a) sufentanil, and panel b) etonitazene.

### EMBODIMENTS

The following non-limiting embodiments provide illustrative examples of the invention, but do not limit the scope of the invention.

### Embodiment 1

A method of treating opioid addiction in a subject in need thereof, the method comprising: implanting into the subject a device comprising a particulate of at least one opioid receptor ligand, or a pharmaceutically-acceptable salt thereof, and a pharmaceutically-acceptable carrier, wherein the particulate has a mean particle size of about 5 μM to about 350 μM, and wherein the device releases a therapeutically-effective amount of the opioid receptor ligand or the pharmaceutically-acceptable salt thereof.

### Embodiment 2

The method of Embodiment 1, wherein the opioid receptor ligand is an opiate, or a pharmaceutically-acceptable salt thereof

### Embodiment 3

The method of any one of Embodiments 1 and 2, wherein the opioid receptor ligand is a synthetic opioid, or a pharmaceutically-acceptable salt thereof.

### Embodiment 4

The method of any one of Embodiments 1-3, wherein the opioid receptor ligand is a semi-synthetic opioid, or a pharmaceutically-acceptable salt thereof.

### Embodiment 5

The method of any one of Embodiments 1-4, wherein the opioid receptor ligand is a partial opioid agonist, or a pharmaceutically-acceptable salt thereof.

### Embodiment 6

The method of any one of Embodiments 1-5, wherein the opioid receptor ligand is buprenorphine or a pharmaceutically-acceptable salt thereof.

### Embodiment 7

The method of any one of Embodiments 1-6, wherein the opioid receptor ligand is a metabolite of buprenorphine, or a pharmaceutically-acceptable salt thereof

### Embodiment 8

The method of Embodiment 7, wherein the metabolite is norbuprenorphine, or a pharmaceutically-acceptable salt thereof

### Embodiment 9

The method of any one of Embodiments 1-8, wherein the particulate is in an amorphous state.

### Embodiment 10

The method of any one of Embodiments 1-9, wherein the particulate is in a solid state.

### Embodiment 11

The method of any one of Embodiments 1-10, wherein the mean particle size is from about 180 μm to about 350 μm.

### Embodiment 12

The method of any one of Embodiments 1-11, wherein the mean particle size is from about 50 μm to about 180 μm.

### Embodiment 13

The method of any one of Embodiments 1-12, wherein the mean particle size is about 10 μm in size.

### Embodiment 14

The method of any one of Embodiments 1-13, wherein the device provides in the subject a plasma concentration of the opioid receptor ligand, or the pharmaceutically-acceptable salt thereof, from about 100 pg/mL to about 900 pg/mL.

### Embodiment 15

The method of any one of Embodiments 1-14, wherein the device provides a plasma concentration of the opioid receptor ligand, or the pharmaceutically-acceptable salt thereof, from about 100 pg/mL to about 4,500 pg/mL.

### Embodiment 16

The method of any one of Embodiments 1-15, wherein the device provides a plasma concentration of a metabolite of the opioid receptor ligand, or the pharmaceutically-acceptable salt thereof, from about 20 pg/mL to about 500 pg/mL.

### Embodiment 17

The method of any one of Embodiments 1-16, wherein the pharmaceutically-acceptable carrier is a polymer.

### Embodiment 18

The method of Embodiment 17, wherein the polymer is ethylene-vinyl acetate.

### Embodiment 19

A method of treating opioid addiction in a subject in need thereof, the method comprising implanting into the subject a device comprising an opioid receptor ligand or a pharmaceutically-acceptable salt thereof, and a pharmaceutically-acceptable carrier, wherein the device has a tensile strength in a range of about 10,000 g/cm² to about 110,000 g/cm²; and wherein the device releases a therapeutically-effective amount of the opioid receptor ligand, or the pharmaceutically-acceptable salt thereof.

### Embodiment 20

The method of Embodiment 19, wherein the opioid receptor ligand is buprenorphine or a pharmaceutically-acceptable salt thereof.

### Embodiment 21

The method of any one of Embodiments 19 and 20, wherein the opioid receptor ligand is a metabolite of buprenorphine, or a pharmaceutically-acceptable salt thereof

### Embodiment 22

The method of Embodiment 21, wherein the metabolite is norbuprenorphine, or a pharmaceutically-acceptable salt thereof.

### Embodiment 23

The method of any one of Embodiments 19-22, wherein the tensile strength of the device ranges from about 10,000 g/cm² to about 50,000 g/cm².

### Embodiment 24

The method of any one of Embodiments 19-23, wherein the tensile strength of the device has an average from about 45,000 g/cm² to about 80,000 g/cm².

### Embodiment 25

The method of any one of Embodiments 19-24, wherein the tensile strength of the device ranges from about 75,000 g/cm² to about 110,000 g/cm².

### Embodiment 26

The method of any one of Embodiments 19-25, wherein the device provides a plasma concentration of the opioid receptor ligand from about 100 pg/mL to about 900 pg/mL.

### Embodiment 27

The method of any one of Embodiments 19-26, wherein the device provides a plasma concentration of the opioid receptor ligand from about 100 pg/mL to about 4,500 pg/mL.

### Embodiment 28

The method of any one of Embodiments 19-27, wherein the device provides a plasma concentration of a metabolite of the opioid receptor ligand from about 20 pg/mL to about 500 pg/mL.

### Embodiment 29

The method of Embodiment 28, wherein the metabolite is norbuprenorphine.

### Embodiment 30

The method of any one of Embodiments 19-29, wherein the pharmaceutically-acceptable carrier is a polymer.

### Embodiment 31

The method of Embodiment 30, wherein the polymer is ethylene-vinyl acetate.

### Embodiment 32

A device comprising: at least one opioid receptor ligand, or a pharmaceutically-acceptable salt thereof, and a pharmaceutically-acceptable carrier, wherein the device has a tensile strength in a range of about 10,000 g/cm² to about 110,000 g/cm²; and wherein, upon implantation of the device in a subject, the device releases a therapeutically-effective amount of the opioid receptor ligand, or the pharmaceutically-acceptable salt thereof to the subject.

### Embodiment 33

The device of Embodiment 32, wherein the opioid receptor ligand is buprenorphine or a pharmaceutically-acceptable salt thereof

### Embodiment 34

The device of any one of Embodiments 32 and 33, wherein the opioid receptor ligand is a metabolite of buprenorphine, or a pharmaceutically-acceptable salt thereof

### Embodiment 35

The device of Embodiment 34, wherein the metabolite is norbuprenorphine, or a pharmaceutically-acceptable salt thereof.

### Embodiment 36

The device of any one of Embodiments 32-35, wherein the tensile strength of the device ranges from about 10,000 g/cm² to about 50,000 g/cm².

### Embodiment 37

The device of any one of Embodiments 32-36, wherein the tensile strength of the device has an average from about 45,000 g/cm² to about 80,000 g/cm².

### Embodiment 38

The device of any one of Embodiments 32-37, wherein the tensile strength of the device ranges from about 75,000 g/cm² to about 110,000 g/cm².

### Embodiment 39

The device of any one of Embodiments 32-38, wherein the device provides a plasma concentration of the opioid receptor ligand from about 100 pg/mL to about 900 pg/mL.

### Embodiment 40

The device of any one of Embodiments 32-39, wherein the device provides a plasma concentration of the opioid receptor ligand from about 100 pg/mL to about 4,500 pg/mL.

### Embodiment 41

The device of any one of Embodiments 32-40, wherein the device provides a plasma concentration of a metabolite of the opioid receptor ligand from about 20 pg/mL to about 500 pg/mL.

### Embodiment 42

The device of Embodiment 41, wherein the metabolite is norbuprenorphine.

### Embodiment 43

The device of any one of Embodiments 32-42, wherein the pharmaceutically-acceptable carrier is a polymer.

### Embodiment 44

The device of Embodiment 43, wherein the polymer is ethylene-vinyl acetate.

### Embodiment 45

A device comprising: a particulate of at least one opioid receptor ligand, or a pharmaceutically-acceptable salt thereof, and a pharmaceutically-acceptable carrier, wherein the particulate has a mean particle size of about 5 μM to about 350 μM, and wherein upon implantation of the device into a subject the device releases a therapeutically-effective amount of the opioid receptor ligand or the pharmaceutically-acceptable salt thereof to the subject.

### Embodiment 46

The device of Embodiment 45, wherein the opioid receptor ligand is an opiate, or a pharmaceutically-acceptable salt thereof

### Embodiment 47

The device of any one of Embodiments 45 and 46, wherein the opioid receptor ligand is a synthetic opioid, or a pharmaceutically-acceptable salt thereof.

### Embodiment 48

The device of claim 45, wherein the opioid receptor ligand is a semi-synthetic opioid, or a pharmaceutically-acceptable salt thereof

### Embodiment 49

The device of any one of Embodiments 45-48, wherein the opioid receptor ligand is a partial opioid agonist, or a pharmaceutically-acceptable salt thereof.

### Embodiment 50

The device of any one of Embodiments 45-49, wherein the opioid receptor ligand is buprenorphine or a pharmaceutically-acceptable salt thereof.

### Embodiment 51

The device of any one of Embodiments 45-50, wherein the opioid receptor ligand is a metabolite of buprenorphine, or a pharmaceutically-acceptable salt thereof

### Embodiment 52

The device of Embodiment 51, wherein the metabolite is norbuprenorphine, or a pharmaceutically-acceptable salt thereof.

### Embodiment 53

The device of any one of Embodiments 45-52, wherein the particulate is in an amorphous state.

### Embodiment 54

The device of any one of Embodiments 45-53, wherein the particulate is in a solid state.

### Embodiment 55

The device of any one of Embodiments 45-54, wherein the mean particle size is from about 180 μm to about 350 μm.

### Embodiment 56

The device of any one of Embodiments 45-55, wherein the mean particle size is from about 50 μm to about 180 μm.

### Embodiment 57

The device of any one of Embodiments 45-56, wherein the mean particle size is about 10 μm in size.

### Embodiment 58

The device of any one of Embodiments 45-57, wherein the device provides a plasma concentration of the opioid receptor ligand from about 100 pg/mL to about 900 pg/mL.

### Embodiment 59

The device of any one of Embodiments 45-58, wherein the device provides a plasma concentration of the opioid receptor ligand from about 100 pg/mL to about 4,500 pg/mL.

### Embodiment 60

The device of any one of Embodiments 45-59, wherein the device provides a plasma concentration of the metabolite of the opioid receptor ligand from about 20 pg/mL to about 500 pg/mL.

### Embodiment 61

The device of any one of Embodiments 45-60, wherein the pharmaceutically-acceptable carrier is a polymer.

### Embodiment 62

The device of Embodiment 61, wherein the polymer is ethylene-vinyl acetate.

What is claimed is:

1. A method of treating opioid addiction in a subject in need thereof, the method comprising: implanting into the subject a device comprising a particulate of at least one opioid receptor ligand, or a pharmaceutically-acceptable salt thereof, and a pharmaceutically-acceptable carrier, wherein the particulate has a mean particle size of about 5 μM to about 350 μM, and wherein the device releases a therapeutically-effective amount of the opioid receptor ligand or the pharmaceutically-acceptable salt thereof.

2. The method of claim 1, wherein the opioid receptor ligand is an opiate, or a pharmaceutically-acceptable salt thereof.

3. The method of claim 1, wherein the opioid receptor ligand is a synthetic opioid, or a pharmaceutically-acceptable salt thereof.

4. The method of claim 1, wherein the opioid receptor ligand is a semi-synthetic opioid, or a pharmaceutically-acceptable salt thereof.

5. The method of claim 1, wherein the opioid receptor ligand is a partial opioid agonist, or a pharmaceutically-acceptable salt thereof.

6. The method of claim 1, wherein the opioid receptor ligand is buprenorphine or a pharmaceutically-acceptable salt thereof.

7. The method of claim 1, wherein the opioid receptor ligand is a metabolite of buprenorphine, or a pharmaceutically-acceptable salt thereof.

8. The method of claim 7, wherein the metabolite is norbuprenorphine, or a pharmaceutically-acceptable salt thereof.

9. The method of claim 1, wherein the particulate is in an amorphous state.

10. The method of claim 1, wherein the particulate is in a solid state.

11. The method of claim 1, wherein the mean particle size is from about 180 μm to about 350 μm.

12. The method of claim 1, wherein the mean particle size is from about 50 μm to about 180 μm.

13. The method of claim 1, wherein the mean particle size is about 10 μm in size.

14. The method of claim 1, wherein the device provides in the subject a plasma concentration of the opioid receptor ligand, or the pharmaceutically-acceptable salt thereof, from about 100 pg/mL to about 900 pg/mL.

15. The method of claim 1, wherein the device provides a plasma concentration of the opioid receptor ligand, or the pharmaceutically-acceptable salt thereof, from about 100 pg/mL to about 4,500 pg/mL.

16. The method of claim 1, wherein the device provides a plasma concentration of a metabolite of the opioid receptor ligand, or the pharmaceutically-acceptable salt thereof, from about 20 pg/mL to about 500 pg/mL.

17. The method of claim 1, wherein the pharmaceutically-acceptable carrier is a polymer.

18. The method of claim 17, wherein the polymer is ethylene-vinyl acetate.

19. A method of treating opioid addiction in a subject in need thereof, the method comprising implanting into the subject a device comprising an opioid receptor ligand or a pharmaceutically-acceptable salt thereof, and a pharmaceutically-acceptable carrier, wherein the device has a tensile strength in a range of about 10,000 g/cm² to about 110,000 g/cm²; and wherein the device releases a therapeutically-effective amount of the opioid receptor ligand, or the pharmaceutically-acceptable salt thereof.

20. The method of claim 19, wherein the opioid receptor ligand is buprenorphine or a pharmaceutically-acceptable salt thereof.

21. The method of claim 19, wherein the opioid receptor ligand is a metabolite of buprenorphine, or a pharmaceutically-acceptable salt thereof.

22. The method of claim 21, wherein the metabolite is norbuprenorphine, or a pharmaceutically-acceptable salt thereof.

23. The method of claim 19, wherein the tensile strength of the device ranges from about 10,000 g/cm² to about 50,000 g/cm².

24. The method of claim 19, wherein the tensile strength of the device has an average from about 45,000 g/cm² to about 80,000 g/cm².

25. The method of claim 19, wherein the tensile strength of the device ranges from about 75,000 g/cm² to about 110,000 g/cm².

26. The method of claim 19, wherein the device provides a plasma concentration of the opioid receptor ligand from about 100 pg/mL to about 900 pg/mL.

27. The method of claim 19, wherein the device provides a plasma concentration of the opioid receptor ligand from about 100 pg/mL to about 4,500 pg/mL.

28. The method of claim 19, wherein the device provides a plasma concentration of a metabolite of the opioid receptor ligand from about 20 pg/mL to about 500 pg/mL.

29. The method of claim 28, wherein the metabolite is norbuprenorphine.

30. The method of claim 19, wherein the pharmaceutically-acceptable carrier is a polymer.

31. The method of claim 30, wherein the polymer is ethylene-vinyl acetate.

32. A device comprising: at least one opioid receptor ligand, or a pharmaceutically-acceptable salt thereof, and a pharmaceutically-acceptable carrier, wherein the device has a tensile strength in a range of about 10,000 g/cm² to about 110,000 g/cm²; and wherein, upon implantation of the device in a subject, the device releases a therapeutically-effective amount of the opioid receptor ligand, or the pharmaceutically-acceptable salt thereof to the subject.

33. The device of claim 32, wherein the opioid receptor ligand is buprenorphine or a pharmaceutically-acceptable salt thereof.

34. The device of claim 32, wherein the opioid receptor ligand is a metabolite of buprenorphine, or a pharmaceutically-acceptable salt thereof.

35. The device of claim 34, wherein the metabolite is norbuprenorphine, or a pharmaceutically-acceptable salt thereof.

36. The device of claim 32, wherein the tensile strength of the device ranges from about 10,000 g/cm² to about 50,000 g/cm².

37. The device of claim 32, wherein the tensile strength of the device has an average from about 45,000 g/cm² to about 80,000 g/cm².

38. The device of claim 32, wherein the tensile strength of the device ranges from about 75,000 g/cm² to about 110,000 g/cm².

39. The device of claim 32, wherein the device provides a plasma concentration of the opioid receptor ligand from about 100 pg/mL to about 900 pg/mL.

40. The device of claim 32, wherein the device provides a plasma concentration of the opioid receptor ligand from about 100 pg/mL to about 4,500 pg/mL.

41. The device of claim 32, wherein the device provides a plasma concentration of a metabolite of the opioid receptor ligand from about 20 pg/mL to about 500 pg/mL.

42. The device of claim 41, wherein the metabolite is norbuprenorphine.

43. The device of claim 32, wherein the pharmaceutically-acceptable carrier is a polymer.

44. The device of claim 43, wherein the polymer is ethylene-vinyl acetate.

45. A device comprising: a particulate of at least one opioid receptor ligand, or a pharmaceutically-acceptable salt thereof, and a pharmaceutically-acceptable carrier, wherein the particulate has a mean particle size of about 5 μM to about 350 μM, and wherein upon implantation of the device into a subject the device releases a therapeutically-effective amount of the opioid receptor ligand or the pharmaceutically-acceptable salt thereof to the subject.

46. The device of claim 45, wherein the opioid receptor ligand is an opiate, or a pharmaceutically-acceptable salt thereof.

47. The device of claim 45, wherein the opioid receptor ligand is a synthetic opioid, or a pharmaceutically-acceptable salt thereof.

48. The device of claim 45, wherein the opioid receptor ligand is a semi-synthetic opioid, or a pharmaceutically-acceptable salt thereof.

49. The device of claim 45, wherein the opioid receptor ligand is a partial opioid agonist, or a pharmaceutically-acceptable salt thereof.

50. The device of claim 45, wherein the opioid receptor ligand is buprenorphine or a pharmaceutically-acceptable salt thereof.

51. The device of claim 45, wherein the opioid receptor ligand is a metabolite of buprenorphine, or a pharmaceutically-acceptable salt thereof.

52. The device of claim 51, wherein the metabolite is norbuprenorphine, or a pharmaceutically-acceptable salt thereof.

53. The device of claim 45, wherein the particulate is in an amorphous state.

54. The device of claim 45, wherein the particulate is in a solid state.

55. The device of claim 45, wherein the mean particle size is from about 180 μm to about 350 μm.

56. The device of claim 45, wherein the mean particle size is from about 50 μm to about 180 μm.

57. The device of claim 45, wherein the mean particle size is about 10 μm in size.

58. The device of claim 45, wherein the device provides a plasma concentration of the opioid receptor ligand from about 100 pg/mL to about 900 pg/mL.

59. The device of claim 45, wherein the device provides a plasma concentration of the opioid receptor ligand from about 100 pg/mL to about 4,500 pg/mL.

60. The device of claim 45, wherein the device provides a plasma concentration of the metabolite of the opioid receptor ligand from about 20 pg/mL to about 500 pg/mL.

61. The device of claim 45, wherein the pharmaceutically-acceptable carrier is a polymer.

62. The device of claim 61, wherein the polymer is ethylene-vinyl acetate.
